(19) United States
(12) Patent Application Publication
(10) Pub. No.: US 2025/0152241 A1
(43) Pub. Date: May 15, 2025

(54) SYSTEMS AND METHODS FOR PLANNING BONE GRAFTS

(71) Applicant: Zimmer, Inc., Warsaw, IN (US)

(72) Inventors: Philippe Favre, Zurich (CH); Jeffrey E. Bischoff, Holland, MI (US); Ghislain Maquer, Zurich (CH); Ingmar Fleps, Boston, MA (US)

(21) Appl. No.: 18/943,455

(22) Filed: Nov. 11, 2024

Related U.S. Application Data
(60) Provisional application No. 63/598,796, filed on Nov. 14, 2023.

(51) Int. Cl. A61B 34/00; A61B 34/10

(52) U.S. Cl. CPC A61B 34/00; A61B 2034/107

(57) ABSTRACT

A computer-implemented method of pre-operatively evaluating bone structure for use of a bone graft in a surgical procedure can comprise generating a three-dimensional bone model of a bone of a patient for output in a video display unit, determining a volume of interest (VOI) on the three-dimensional bone model where a bone defect exists, adding an internal bone property for bone matter of the bone structure to the three-dimensional bone model at the volume of interest, analyzing the internal bone property in the volume of interest relative to a treatment for a bone defect, and outputting indicia on the video display unit that indicates utility of a bone graft for the treatment. A surgical planning system can comprise memory having instructions for implementing the computer-implemented method.

### CLAIM OF PRIORITY

This application claims the benefit of U.S. Provisional Patent Application Ser. No. 63/598,796, filed on Nov. 14, 2023, the benefit of priority of which is claimed hereby, and which is incorporated by reference herein in its entirety.

### TECHNICAL FIELD

The present disclosure is directed to systems, devices and methods for use in planning and performing surgical procedures, such as those using implants to be affixed in bone as in the case of orthopedic and trauma surgeries. More particularly, the present disclosure is directed to the use of bone graft material that can be used to augment or supplant native bone matter at or near the implantation site of the implant.

### BACKGROUND

Bone grafting involves the replacement or augmenting of unhealthy, low-density or missing bone matter with a substitute bone material. The bone graft can be from another species (heterologous), synthetic material, or from the same patient (autologous graft). The advantages of autologous bone grafts are their excellent biologic compatibility and similar mechanical properties with the grafting site compared to other grafting techniques, but they result in considerable donor site morbidity and the volume available at the harvesting sites is limited. Autologous bone graft material can include trabecular (cancellous) bone, cortical bone and marrow, individually or in combination.

Bone grafting can be used in orthopedic and trauma surgical procedures for joint fusion, regeneration (after bone loss due to disease, infection, cancer, injury, etc.), or to improve fixation around an implanted device. Orthopedic procedures often involve the implantation of a prosthetic component, such as a humeral head. Trauma procedures often involve the implantation of a bone plate or nail to hold together fragments of a broken bone. Bone grafting is also a regular procedure in dental surgery, and plastic and reconstructive surgery. In case of insufficient bone stock where surgery is performed, the surgeon may opt for a bone graft to provide enough support for the implant or for healing and skeletal support.

Pat. Nos. U.S. Pat. Nos. 10,736,539; 10,512,451; 10,258,256; and U.S. Pat. No. 10,130,478 to Mahfouz describe various methods of planning for orthopedic implant procedures.

### OVERVIEW

The present inventors have recognized, among other things, that an issue, or problem to be solved, with traditional bone grafting procedures is that the need for bone grafting or the suitability of a harvest site is only ascertained intra-operatively, e.g., when bone quality of the patient can be directly evaluated at the defect and donor site. The present inventors have recognized that intra-operative evaluation is not optimal and that a preoperative assessment is less likely to be detrimental for the patient. Furthermore, the present inventors have recognized that intra-operative bone graft evaluation typically relies on surgeon experience and judgment and thus, does not always adequately take into account internal bone properties, such as density, strength (e.g., compressive or tensile strength) and stiffness. Thus, even if a suitable volume of bone graft material is taken from the patient intra-operatively, it is not always known if the bone graft material will provide suitable performance in terms of allowing for adequate healing and support.

Grafting procedures often require a surgical procedure at another body part, e.g., the donor or harvest site, than the treated location, e.g., the defect site, to harvest the graft material. Complications occurring after bone grafting include, for example, fracture at the harvest site, intra-operative bleeding, postoperative pain, inflammation, infection. Furthermore, grafting leads to increased surgical time and hospital length of stay with consequent additional costs.

Cortical bone grafts are frequently used in trauma procedures. Generally, for non-vascularized cortical grafts it is desirable to use bone matter from the iliac crest and for vascularized cortical grafts to use bone matter from the fibula. Such locations provide strong donor bone matter from an area of the body that is not typically subject to high loads and can therefore accommodate bone loss. Cancellous or trabecular bone grafts are often used in orthopedic or prosthetic procedures. However, desirable donor sites for cancellous grafts are debatable. With the “Reamer Irrigator Aspirator” (RIA) System, large quantities of autologous bone graft can be harvested from the femoral and tibial medullary cavities, each site being associated with specific potential complications. Examples of RIA systems are described in Pub. No. US 2021/0153877 to Kiersh et al., Pub. No. US 2022/0202992 to Lehmicke et al. and Pat. No. U.S. Pat. No. 10,646,235 to Kiersh et al.

In current clinical practice for both cortical and cancellous bone grafts, the surgeon does not know pre-operatively if a bone graft will be required for sure, and if the quality and quantity of bone at a particular donor site will be sufficient for the intended use. The size, e.g., volume and shape, and mass or density of the bone graft should be proper for the bone defect; otherwise, the reduction will not be anatomically correct. The surgeon will currently have this information intra-operatively, only after harvesting the graft. This puts the success of the bone graft at risk, and the surgeon may have to perform multiple grafts, thereby significantly increasing the risk of complication. As understood by the present inventors, there does not currently exist a tool or system to allow the surgeon to evaluate pre-operatively if a defect site will require a bone graft and if a harvest site will yield a sufficiently large, dense or strong bone graft.

Problems associated with not knowing bone density and strength or improperly assessing bone density and strength for one or both the defect site and the harvest site involve 1) selecting suboptimal locations for altering the bone at the defect site (e.g., locations where unhealthy or less dense bone engages the orthopedic implant); 2) selecting suboptimal locations for the harvest site; 3) damaging the bone of the patient at the defect and harvest sites during bone altering procedures (e.g., cutting, drilling, resecting); and 4) adverse post-operative effects associated with insufficient bony support of the implant (e.g., loosening, stress shielding, fracture and dissociation).

The present subject matter can provide solutions to these and other problems, such as by providing systems, devices and methods for surgical planning that allow surgeons or surgical planners to obtain an indication of bone size, density and strength at a defect site and at one or more potential harvest sites pre-operatively or intra-operatively as well as providing suitability of bone size, density and strength at the defect site and the harvest site for desirable outcomes of the procedure. The solutions can include one or more of the following options: use of surgery planning systems and software to A) obtain three-dimensional bone size, density and strength information at the defect site and one or more harvest sites; B) run pre-operative and intra-operative procedure simulations to evaluate bone graft donor material; C) run artificial-intelligence-powered models to determine defect site volumes of interest and harvest site volumes of interest and to evaluate bone size, density and strength at defect sites and potential harvest sites; D) to evaluate different risk factors based on bone density and strength information; and E) develop robotic positioning information (the location of various cut planes within the bone to form the defect site and obtain the harvested bone graft material, etc.) and patient-specific instrumentation correlated to the defect site volume of interest and the harvest sites volumes of interest.

In an example, a computer-implemented method of pre-operatively evaluating bone structure for use of a bone graft in a surgical procedure can comprise generating a three-dimensional bone model of a bone of a patient for output in a video display unit, determining a volume of interest (VOI) on the three-dimensional bone model where a bone defect exists, adding an internal bone property for bone matter of the bone structure to the three-dimensional bone model at the volume of interest, analyzing the internal bone property in the volume of interest relative to a treatment for a bone defect, and outputting indicia on the video display unit that indicates utility of a bone graft for the treatment.

In another example, a surgical planning system can comprise a controller for a computer-implemented surgical planning system, an output device in communication with the controller, an input device in communication with the controller, and memory having instructions stored therein executable by the controller to generate a surgical plan, the instructions comprising generating a three-dimensional bone model showing bone structure of a bone of a patient for output in the output device, determining a first volume of interest (VOI) on the three-dimensional bone model where a bone defect exists, determining a second volume of interest (VOI) on the three-dimensional bone model where a potential bone graft exits, adding an internal bone property for bone matter of the bone structure to the three-dimensional bone model at the first VOI and the second VOI, analyzing the internal bone property in the volume of interest relative to a treatment for a bone defect, and outputting indicia on the output device that indicates utility of a bone graft for the treatment.

### BRIEF DESCRIPTION OF THE DRAWINGS

FIG. 1 is a diagrammatic view of an operating room including a robot-assisted surgical system comprising a robotic arm, a computing system, and a tracking system with which the systems, devices and methods of the present disclosure can be implemented.

FIG. 2 is a partial cross-sectional view of the humeral side of an anatomic total shoulder system comprising a prosthetic humeral head fixed to a stem implanted into cancellous and cortical bone material of a humerus.

FIGS. 3A and 3B are perspective top and bottom views of a stemless humeral implant showing fixation blades for cancellous bone and a coupler for a humeral head prosthesis that replaces the native humeral head.

FIG. 4 is a schematic side view of a humeral bone having a trauma plate attached to the cortical shell using a plurality of screws.

FIG. 5A is a schematic front view of a humeral bone and a scapula bone of a shoulder joint positioned within in imaging system.

FIG. 5B is a schematic view of an x-ray image of the shoulder joint of FIG. 5A with a templating image of a stemless humeral implant and a bone density scale.

FIGS. 6A-6D schematically illustrate various operations in methods of evaluating a bone graft for trabecular bone matter using modeling and simulating systems of the present disclosure.

FIG. 7 is a block diagram illustrating operations of methods for evaluating and performing a bone graft procedure for trabecular bone matter.

FIGS. 8A-8D schematically illustrate various operations in methods of evaluating a bone graft for cortical bone matter using modeling and simulating systems of the present disclosure.

FIG. 9 is a block diagram illustrating operations of methods for evaluating and performing a bone graft procedure for cortical bone matter.

FIG. 10 is a schematic illustration of a surgical planning system incorporating a simulation engine and an artificial intelligence engine for evaluating bone strength and density information and evaluating bone graft defect sites and bone graft harvest sites using bone density information.

FIG. 11 is a block diagram of an example machine upon which any one or more of the techniques and methods discussed herein can be performed and with which any of the devices discussed herein can be used in accordance with some embodiments.

### DETAILED DESCRIPTION

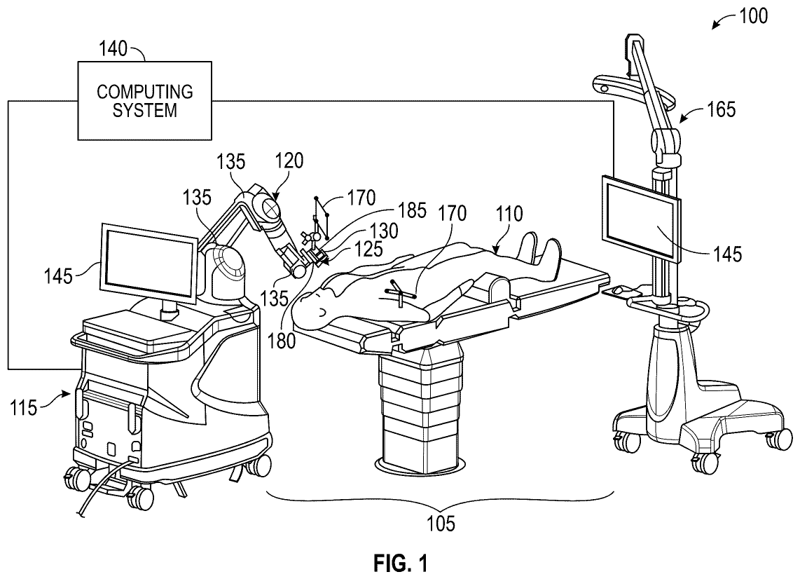

FIG. 1 illustrates surgical system 100 for operation on surgical area 105 of patient 110 in accordance with at least one example of the present disclosure. In examples, surgical area 105 can include a joint, including one or more bones. Surgical area 105 can include any surgical area of patient 110, including but not limited to the shoulder, head, elbow, hip, ankle, thumb, spine, and the like. Surgical system 100 can also include robotic system 115 with one or more robotic arms, such as robotic arm 120. As illustrated, robotic system 115 can utilize only a single robotic arm. Robotic arm 120 can be a 6 degree-of-freedom (DOF) robot arm, such as the ROSA® robot from Medtech, a Zimmer Biomet Holdings, Inc. company. In some examples, robotic arm 120 can be cooperatively controlled with surgeon input on the end effector or surgical instrument, such as surgical instrument 125. In other examples, robotic arm 120 can operate autonomously. While not illustrated in FIG. 1, one or more positionable surgical support arms can be incorporated into surgical system 100 to assist in positioning and stabilizing instruments or anatomy during various procedures.

Each robotic arm 120 can rotate axially and radially and can receive a surgical instrument, or end effector, 125 at distal end 130. Surgical instrument 125 can be any surgical instrument adapted for use by the robotic system 115, including, for example, a guide tube, a holder device, a gripping device such as a pincer grip, a burring device, a reaming device, an impactor device such as a humeral head impactor, a pointer, a force-limiting device, a probe or the like. Surgical instrument 125 can be positioned by robotic arm 120, which can include multiple robotic joints, such as joints 135, that allow surgical instrument 125 to be positioned at any desired location adjacent or within a given surgical area 105. Robotic arm 120 can be used with an instrument positioning device, e.g., an instrument holder, to position an instrument in a known, desired, or predetermined orientation relative to surgical area 105 based on a virtual coordinate system determined by computing system 140.

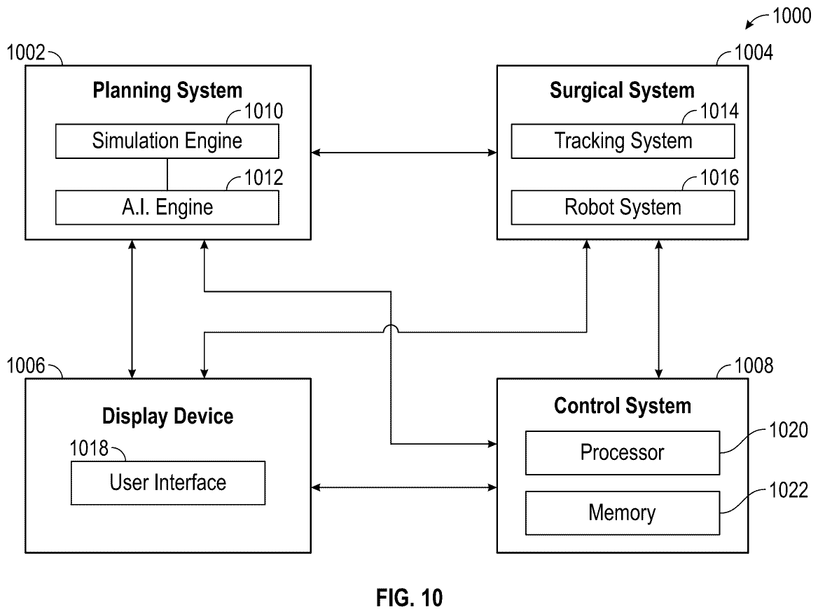

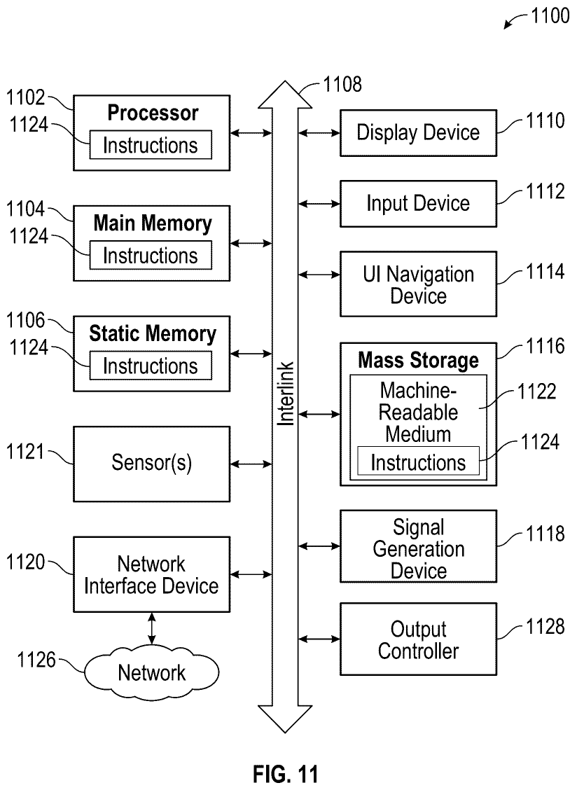

Robotic system 115 can also include computing system 140 that can operate robotic arm 120 and surgical instrument 125. Computing system 140 can include at least memory, a processing unit, and user input devices, as will be described herein, such as with reference to FIGS. 10 and 11. Computing system 140 and tracking system 165 can also include human interface devices 145 for providing images for a surgeon to be used during surgery. Computing system 140 is illustrated as a separate standalone system, but in some examples computing system 140 can be integrated into robotic system 115. Human interface devices 145 can provide images, including but not limited to three-dimensional images of bones, joints, prostheses, and the like, as well as bone density information for generic bones and specific bones of a patient. Human interface devices 145 can include associated input mechanisms, such as a touch screen, foot pedals, or other input devices compatible with a surgical environment.

Computing system 140 can receive pre-operative, intra-operative and post-operative medical images. These images can be received in any manner and the images can include, but are not limited to, computed tomography (CT) scans, magnetic resonance imaging (MRI), x-rays, ultrasound, and the like. As discussed herein, these images can include, or can be modified to include, bone strength and density information that can be used to, for example, produce three-dimensional models of anatomy of a specific patient that can indicate the need for bone grafts and the suitability of bone graft harvest sites. The images and three-dimensional bone density models, in examples, can be sent via a server, such as a file transfer protocol, cloud storage or as files attached to an email or other electronic communication means. In another example the images can be stored on an external memory device such as a memory stick and coupled to a USB port of the robotic system to be uploaded into the processing unit. In yet other examples, the images can be accessed over a network by computing system 140 from a remote storage device or service.

After receiving one or more images, computing system 140 can generate one or more virtual models related to surgical area 105, such as a three-dimensional model incorporating, for example, bone strength and density information or anatomy information. Alternatively, computing system 140 can receive virtual models of the anatomy of the patient prepared remotely. Specifically, a virtual model of the anatomy of patient 110 can be created by defining anatomical points within the image(s) and/or by fitting a statistical anatomical model to the image data. A 3D model can comprise a mathematical coordinate-based representation of any surface of an object in three dimensions via specialized software by manipulating edges, vertices, and polygons in a simulated 3D space. A virtual model can comprise a 3D model augmented with anatomic information, e.g., bone density information, from a patient. The bone density information can be derived from various sources including images used to generate the 3D model. In examples, the virtual model can comprise a finite element model. Additionally, the virtual model can be created using multiple medical images, such as x-ray images, of the anatomy of the patient merged together to form a three-dimensional model. The virtual model, along with virtual representations of implants, can be used for calculations related to the desired height, depth, inclination angle, or version angle of an implant, stem, surgical instrument, or the like related to be utilized in surgical area 105. In another procedure type, the virtual model can be utilized to determine insertion location, trajectory, insertion force (e.g., an insertion force ceiling to avoid adversely affecting bone structure of a specific patient) and depth for inserting an instrument. The virtual model can also be used to determine bone dimensions, implant dimensions, bone fragment dimensions, bone fragment arrangements, and the like. Any model generated, including three-dimensional models, can be displayed on human interface devices 145 for reference during a surgery or used by robotic system 115 to determine motions, actions, and operations of robotic arm 120 or surgical instrument 125. Known techniques for creating virtual bone models can be utilized, such as those discussed in U.S. Pat. No. 9,675,461, titled “Deformable articulating templates” or U.S. Pat. No. 8,884,618, titled “Method of generating a patient-specific bone shell” both by Mahfouz, as well as other techniques known in the art.

The patient-specific, three-dimensional bone models (e.g., virtual models) can be used to, for example, 1) determine volumes of bone graft sites; 2) determine bone density within the bone graft sites; 3) determine bone strength within the bone graft sites; 4) determine locations for bone modifications in order to prepare a bone to receive a prosthetic device accounting for the density and strength of the bone matter and the need or desirability for a bone graft; and 5) determine robotic surgical system coordinates and patient-specific instruments based on, for example, bone density and strength at the bone graft site to facilitate harvesting or obtaining the bone graft donor material.

Additional relevant patient-specific information can include demographic data, such as age, gender, ethnicity, height, weight, but also information such as activity level, medication and health care, mental status and the like. This information is available in the planning phase, is known to influence bone quality, and can be used directly as additional information to the planning step using AI.

Computing system 140 can also communicate with tracking system 165 that can be operated by computing system 140 as a stand-alone unit. Surgical system 100 can utilize the Polaris optical tracking system from Northern Digital, Inc. of Waterloo, Ontario, Canada. Additionally, tracking system 165 can comprise the tracking system shown and described in Pub. No. US 2017/0312035, titled “Surgical System Having Assisted Navigation” to Brian M. May, which is hereby incorporated by this reference in its entirety. Tracking system 165 can monitor a plurality of tracking elements, such as tracking elements 170, affixed to objects of interest to track locations of multiple objects within the surgical field. Tracking system 165 functions to create a virtual three-dimensional coordinate system within the surgical field for tracking patient anatomy, surgical instruments, or portions of robotic system 115. Tracking elements 170 can be tracking frames including multiple IR reflective tracking spheres, or similar optically tracked marker devices. In one example, tracking elements 170 can be placed on or adjacent one or more bones of patient 110. In other examples, tracking elements 170 can be placed on robotic arm 120, surgical instrument 125, and/or an implant to accurately track positions within the virtual coordinate system associated with surgical system 100. In each instance tracking elements 170 can provide position data, such as patient position, bone position, joint position, robotic arm position, implant position, or the like.

Robotic system 115 can include various additional sensors and guide devices. For example, robotic system 115 can include one or more force sensors, such as force sensor 180. Force sensor 180 can provide additional force data or information to computing system 140 of robotic system 115. Force sensor 180 can be used by a surgeon to cooperatively move robotic arm 120. For example, force sensor 180 can be used to monitor impact or implantation forces during certain operations, such as insertion of an implant stem into an intramedullary canal. Monitoring forces can assist in preventing negative outcomes through force fitting components. In other examples, force sensor 180 can provide information on soft-tissue tension in the tissues surrounding a target joint. In certain examples, robotic system 115 can also include laser pointer 185 that can generate a laser beam or array that is used for alignment of implants during surgical procedures.

In order to ensure that computing system 140 is moving robotic arm 120 in a known and fixed relationship to surgical area 105 and patient 110, the space of surgical area 105 and patient 110 can be registered to computing system 140 via a registration process involving registering fiducial markers attached to patient 110 with corresponding images of the markers in patient 110 recorded pre-operatively or just prior to a surgical procedure. For example, a plurality of fiducial markers can be attached to patient 110, images of patient 110 with the fiducial markers can be taken or obtained and stored within a memory device of computing system 140. Subsequently, patient 110 with the fiducial markers can be moved into, if not already there because of the imaging, surgical area 105 and robotic arm 120 can touch each of the fiducial markers. Engagement of each of the fiducial markers can be cross-referenced with, or registered to, the location of the same fiducial marker in the images. In additional examples, patient 110 and medical images of the patient can be registered in real space using contactless methods, such as by using a laser rangefinder held by robotic arm 120 and a surface matching algorithm that can match the surface of the patient from scanning of the laser rangefinder and the surface of the patient in the medical images. As such, the real-world, three-dimensional geometry of the anatomy attached to the fiducial markers can be correlated to the anatomy in the images and movements of surgical instruments 125 attached to robotic arm 120 based on the images will correspondingly occur in surgical area 105.

Subsequently, other instruments and devices attached to surgical system 100 can be positioned by robotic arm 120 into a known and desired orientation relative to the anatomy to implant a selected prosthesis according to the selected operative plan. The location where robotic arm 120 holds instruments and where associated resections or bone alterations are placed on a bone can be planned using patient-specific soft tissue information. In examples, surgical system can position robotic arm 120 into positions to obtain bone graft material from harvest sites, directing the appropriate instruments, e.g., cutting guides, to the harvest site and to make the corresponding appropriate cuts to obtain the desired volume of material from the harvest site.

As discussed herein, surgical system 100 and in particular computing system 140 can be used to provide pre-operative or intra-operative information, evaluation and feedback regarding bone volume, density, and strength at a defect site and at one or more potential harvest sites. The feedback can comprise discrete information about the bone size, density and strength at specific locations in the bone matter. The feedback can also comprise information, such as comparison to database information regarding suitable or acceptable bone densities and strengths for defect locations and harvest locations. The feedback can be in the form of visual indicators, such as heat maps, risk scales, color coding and the like to inform a surgical team about specific risks or suitability for a specific patient or bone graft. Furthermore, the feedback can comprise discrete recommendations, such as no-go or go recommendations for bone defect sites and bone graft sites.

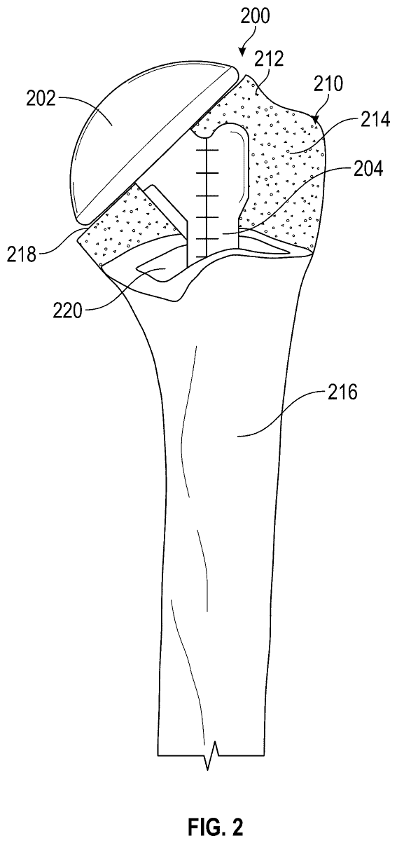

FIG. 2 is a partial cross-sectional view of humeral prosthesis 200 having head 202 and stem 204. The humeral prosthesis 200 can be implanted into humerus 210. A portion of humerus 210 is cut-away in FIG. 2 to show stem 204 and intramedullary canal 220. In examples, humeral prosthesis 200 can comprise the prosthetic device described in Pat. No. U.S. Pat. No. 10,925,738, titled “Adjustable Orthopedic Connections” to Nathan A. Winslow et al., the contents of which are hereby incorporated by reference. Though the present application is discussed with reference to bones of a shoulder joint, the systems, devices and methods of the present disclosure can be used to in conjunction with other bones and joints, such as ankle, knee and hip joints.

Humerus 210 can comprise base 212 where a humeral head has been resected, tubercle area 214 and diaphysis region 216. Humerus 210, as every long bone, is comprised of a sponge-like interior called cancellous (trabecular) bone surrounded by a stiff cortical shell. Humeral head 212 can be resected to form cut surface 218 that can expose intramedullary canal 220. Humeral prosthesis 200 can be attached to humerus 210 via insertion of stem 204 into intramedullary canal 220 until head 202 contacts or is in close proximity to cut surface 218.

Head 202 and stem 204 can be fabricated of typical materials for prosthetic implants, such as titanium or stainless steel. Such materials can be hard and as such are desirable to reduce wear and prevent damage or corrosion. However, such hard materials can be significantly harder than the bone material to which they are attached. As such, there is the potential for stem 204 to damage humerus 210, particularly during the implant procedures. To implant humeral prosthesis 200, intramedullary canal 220 can be reamed to produce a cavity to receive stem 204. The cavity can be produced to be slightly smaller than stem 204 in order to obtain a press-fit so that humeral prosthesis 200 is not loose and less likely to shift position.

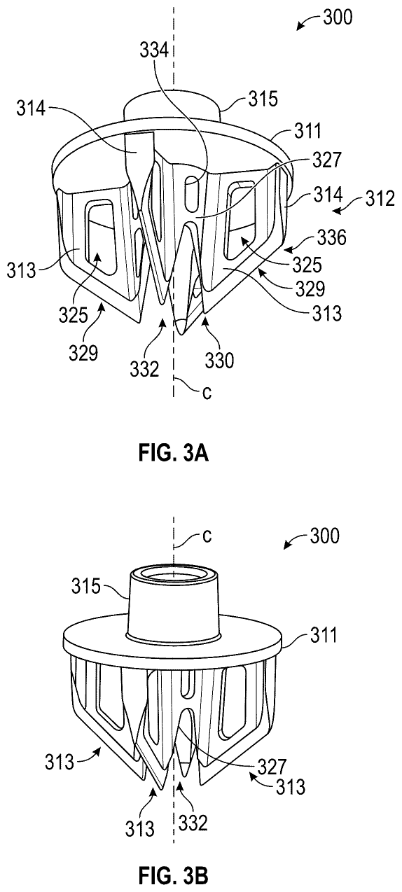

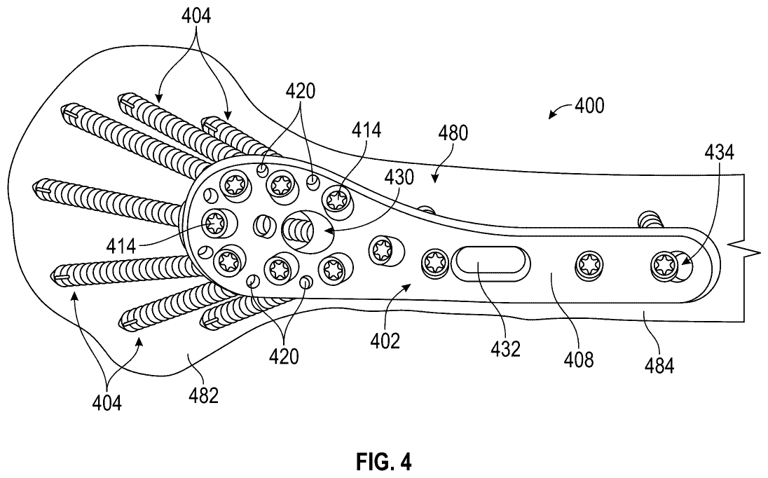

The surgical planning and predicting systems and methods of the present disclosure can pre-operatively analyze patient-specific bone density and strength information in particular volumes of interest (VOI) of bone matter to help determine if a bone graft is to be used at an implantation or defect site for a prosthesis or a graft site for a bone repair and, if a bone graft is determined to be used, select a harvest site that will yield bone graft material of suitable density and strength. For example, the surgical planning and predicting systems and methods of the present disclosure can determine bone density of cancellous bone matter to facilitate evaluation of an orthopedic procedure for an orthopedic implant, such as the humeral head prosthesis shown in FIGS. 3A and 3B, or bone strength of cortical bone matter to facilitate evaluation of a trauma procedure, such as the bone plate shown in FIG. 4, or both.

FIG. 3A is a perspective view of stemless anchor 300, which can be attached to a humeral head prosthesis (not shown in FIG. 3A). Stemless anchor 300 can comprise base plate 311 and blades 313. In examples, stemless anchor 300 can comprise the humeral head implant described in Pat. No. U.S. Pat. No. 8,992,623, titled “Shoulder Prosthesis” to Andrew Hopkins et al., the contents of which are hereby incorporated by reference.

Stemless anchor 300 can comprise base plate 311, which can comprise a circular disk having a first proximal side and a second distal side connected by an edge surface or rim. Connection portion 315 can extend from the first side of base plate 311 in a first direction and blades 313 can extend from a second side of base plate 311 in a second direction. Connection portion 315 can comprise a stud or receptacle for attaching a prosthetic humeral head (e.g., head 202 of FIG. 2) to base plate 311. In examples, connection portion 315 can comprise a tapered cylinder-like body. However, connection portion 315 can comprise any suitable connection means to reliably connect the humeral head to stemless anchor 300.

The distal side of base plate 311 can be provided with anchoring means 312 that serves to reliably anchor stemless anchor 300 in the humerus bone of a patient. Anchoring means 312 can comprise blades 313 extending from base plate 311. In examples, four blades 313 can be used. However, in other examples, a greater number of blades 313 or a lesser number of blades 313 can be used. In examples, blades 313 can comprise ribs or shanks having planar sidewalls that extend perpendicularly from base plate 311. In examples, blades 313 can be evenly distributed in a circumferential direction around central axis C. In examples having four blades 313, the angle between neighboring blades 313 can be 90°. However, other angles can be provided between neighboring blades 313 and the angles between all blades 313 need not be equal. Blades 313 can extend in a radial direction from central axis C. In examples of the present disclosure, the configuration of prosthetic implants can be customized for specific patients. For example, the number and orientation of blades 313 can be selected based on bone density information of a specific patient.

Blades 313 can be provided with openings 325. Openings 325 can improve blood circulation and osseointegration of stemless anchor 300. Moreover, openings 325 and space F can help minimize the size of stemless anchor 300 thereby minimizing the surgical impact of stemless anchor 300 while at same time promoting osseointegration.

The radially inner ends 330 of neighboring blades 313 can be connected by webs 327. Webs 327 can extend distally from base plate 311 and can have lengths shorter than blades 313. Webs 327 can enclose central space 332 adjacent to base plate 311 that is free of protrusions. Therefore, in an implanted state of stemless anchor 300, material of the humerus bone can extend into central space 332 promoting osseointegration. To foster this process, webs 327 can be provided with openings 334 which can, among other things, improve blood circulation in regions adjacent to stemless anchor 300. In particular, openings 334 can allow circulation of blood into and out of the bony material disposed in central space 332.

The radially outer ends 336 of blades 313 can be provided with wings 314 that extend in in a circumferential direction. In examples, wings 314 can be disposed flush with the outer contour of base plate 311. Wings 314 can improve anchoring properties of blades 313 and contribute, as with webs 327, to the stability and stiffness of stemless anchor 300.

The distal edges of wings 314 and blades 313 can form cutting edges 329 that can facilitate implantation of stemless anchor 300. Distal edges of webs 327 are illustrated as not having cutting edges, but can be provided with cutting edges in other examples.

The geometry of blades 313 can be such that stemless anchor 300 resembles, in a side view, the shape of an arrowhead, i.e., the distal edges of blades 313 can recede in a radial direction towards the radially outer end 336 of blades 313. The edges of radially inner ends 330 of blades 313 can be inclined with respect to central axis C. In other words, the radially inner edges of blades 313 diverge when viewed along central axis C from a distal surface of base plate 311, i.e., when viewed from proximal to distal. Central space 332 can, therefore, have a conical shape tapering towards base plate 311.

Humeral prosthesis 200 (FIG. 2) and stemless anchor 300 (FIGS. 3A and 3B) can have different bone matter footprints and can therefore require different modification be made to humerus 210 (FIG. 2). Humeral prosthesis 200 and stemless anchor 300 will therefore interact with different bone matter of humerus 210, which can potentially have different densities. The differing bone matter density at different locations within humerus 210 can affect how well each of humeral prosthesis 200 and stemless anchor 300 will attach to bone and how well each will stay attached post-operatively, particularly based on the activity of each specific patient.

The systems, devices and methods of the present disclosure can determine a volume of interest for different types of prosthesis depending on how the anchor portion, e.g., blades 313 or stem 204, of each prosthesis interacts with bone matter, and can determine bone density and strength in the volumes of interest to determine if a bone graft is needed for one or both types of implants. Thereafter, the systems, devices and methods of the present disclosure can identify harvest sites on the patient, either on the same bone close to the defect site or on another bone, that have sufficient volume of bone to provide a bone graft of suitable strength and density. The systems, devices and methods of the present disclosure can provide evaluation and objective feedback, such as in the form of visual indicators, regarding how suitable the bone density is in a selected volume of interest.

FIG. 4 is a schematic side view of humeral bone 480 and bone plate assembly 400. Bone plate assembly 400 can comprise bone plate 402 and fasteners 404. Humeral bone 480 can comprise humeral head 482 and humeral shaft 484. Bone plate 402 can comprise outer surface 408 and an inner surface opposite thereto. Holes 414 can comprise threaded bores extending through bone plate 402 from outer surface 408 to the inner surface. Holes 414 can be configured to receive fasteners 404. Holes 420 can pass through bone plate 402 from outer surface 408 to the inner surface. Holes 420 can be configured to receive sutures. Bone plate 402 can be attached to humeral bone 480 in cases where humeral bone 480 is cracked or fractured. In various scenarios, humeral bone 480 can be fractured into pieces that have to be reassembled. Sometimes, the pieces are too small or fragmented to be reassembled or to provide suitable structural support to allow the bone to heal. As such, it can be desirable to obtain a bone graft from a harvest site of a sufficiently large size to fill in where the bone fragments have been separated from. Bone plate 402 can be used to hold the bone fragments and bone grafts in place to allow humeral bone 480 to heal. Bone plate 402 can also include graft hole 430 for injecting osteo-biologics for bone graft applications with a delivery device and elongated threaded slot 432 and opening 434 formed by two overlapping threaded holes to provide options for fasteners 404 to extend along different trajectories. Examples of bone plates suitable for use with the present disclosure are described in Pat. No. U.S. Pat. No. 8,926,675 to Leung et al., Pat. No. U.S. Pat. No. 7,731,718 to Schwammberger et al., and Pat. No. U.S. Pat. No. 7,744,638 to Orbay.

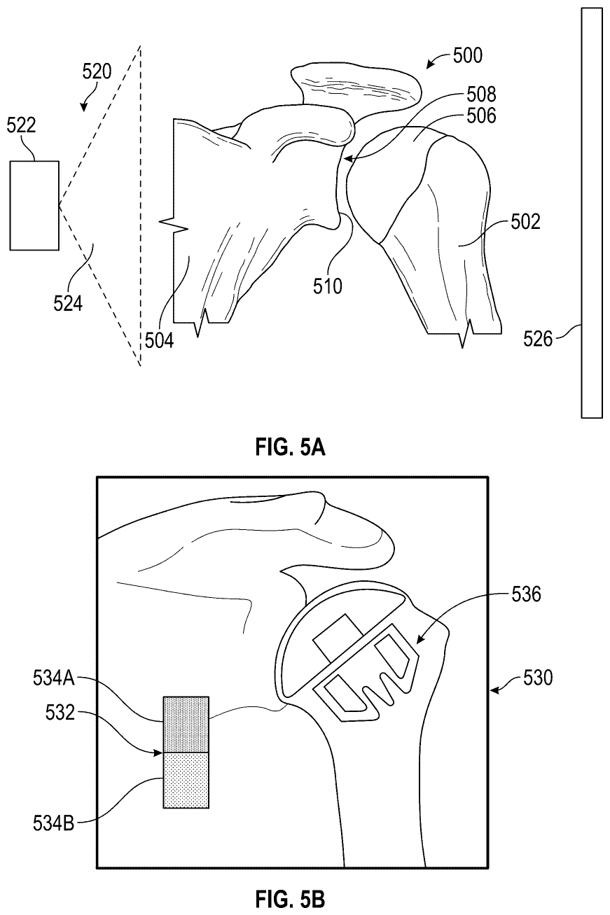

FIG. 5A is a schematic view of shoulder joint 500. Shoulder joint 500 can be of patient 110 of FIG. 1. Shoulder joint 500 can comprise humerus 502 and scapula 504. In FIG. 5A, shoulder joint 500 is illustrated proximate imaging system 520, which can comprise image generator 522, imaging projection 524 and detector 526. FIG. 5B is a schematic view of x-ray image 530 of shoulder joint 500 along with bone density scale 532 and prosthesis template 536. FIGS. 5A and 5B are discussed concurrently.

Humerus 502 of shoulder joint 500 can include head 506, which can have a general ball-shape. Scapula 504 of shoulder joint 500 can include socket or glenoid 508 having glenoid surface 510. During movement of shoulder joint 500, head 506 of humerus 502 can articulate within glenoid 508 of scapula 504 against glenoid surface 510. Though the present application is discussed with reference to bones of shoulder joint 500, the systems, devices and methods of the present disclosure can be used to in conjunction with other bones and joints, such as ankle, knee, and hip joints.

In examples, imaging system 520 can comprise an x-ray imaging system wherein image generator 522 can comprise a high voltage generator x-ray tube, imaging projection 524 can comprise an x-ray beam or field, and detector 526 can comprise an imaging detector, which typically comprises a digital video detector, a solid-state detector, or x-ray film. However, other types of imaging systems can be used. In examples, Magnetic Resonance Imaging (MRI), computed tomography (CT) scans, Dual Energy X-ray Absorptiometry (DEXA or DXA), X-ray, ultrasound imaging systems and the like can be used.

Humerus 502 and scapula 504 can be positioned proximate imaging system 520 within the field of view of image generator 522. Imaging projection 524 emanating from image generator 522 can travel through humerus 502 and scapula 504 to impinge upon detector 526. X-ray image 530 (FIG. 5B) produced by detector 526 can then pass through an amplifier and a computer for processing, or may be recorded on x-ray film in a developer.

In examples, a bone density marker, or a calibration phantom, which contains inserts simulating various trabecular bone densities can be positioned with the field of view of imaging system 520. In examples, bone density scale 532 can be generated using the inserts' mineral densities of a calibration phantom as a reference. Thus, known bone mineral densities of a calibration phantom can be used to compare images of a calibration phantom to images of bone to extrapolate bone mineral density from the native grey values of the CT image. In examples, a calibration phantom can comprise a block of material fabricated from a demineralized bone matrix composite. In examples, the demineralized bone matrix composite can be made according to Pat. No. U.S. Pat. No. 7,582,309 to Rosenberg et al., which is assigned to Etex Corporation. In examples, the demineralized bone matrix composite can be fabricated from Equivabone® Bone Graft Substitute or βBeta-bsm® Injectable Bone Substitute Material, each of which is commercially available from Zimmer Biomet. See US 2022/0183758 A1 to Bailey titled “Patient-specific orthopedic implants and procedures using bone density,” the contents of which are incorporated by this reference. In other examples, bone density scale 532 can comprise a graphic image or icon derived from empirical data that can be included on CT image 530. In additional examples, a bone density calibration phantom can be used, such as those commercially available from QRM of Möhrendorf Germany, a subsidiary of PTW Dosimetry. Calibration phantoms can comprise “solid” phantoms, which based on inserts of various concentrations of calcium hydroxyapatite (HA) in water-equivalent plastic, such as those from QRM, and “liquid” phantoms, based on inserts of various concentrations dipotassium hydrogen phosphate (K2HPO4) solutions. Other potential imaging modalities include Dual Energy X-ray Absorptiometry (DEXA or DXA), which computes bone mineral density intrinsically and is the main tool in the diagnosis and follow-up of osteoporosis. DEXA is a 2D measurement, but 3D density can be simulated with a 2D-3D reconstruction method. CT can obtain 3D bone density with a bone density calibration (BDC) phantom.

X-ray image 530 of shoulder joint 500 can show various internal features of humerus 502 and scapula 504, such as bone structure. Humerus 502 and scapula 504 can have varying bone density from patient to patient, which can be visible in x-ray images and other image types. Bone density is a product of the mineral content in the bone tissue. Bone density can be indirectly determined or estimated by measuring the density per cubic unit of bone within, for example, an CT image. For example, bone matter afflicted with osteoporosis can be less dense than healthy bone and can show up as a lighter grayscale intensity than healthy bone in an x-ray image. Less dense bone can be weaker than healthy bone and is, therefore, more vulnerable to breaking or cracking. For example, hip replacement procedures are particularly vulnerable to bone cracking, either directly from the implant procedure or due to long term fatigue that arises from stresses imparted during the implant procedure. Thus, reduction in the amount of stress imparted to a bone during an implantation procedure can be used to reduce the occurrence of revision surgeries. As such, healthy bone will have a density, e.g., an associated grayscale intensity in x-ray image 530, replicated by shading 534A on bone density scale 532, while diseased or damaged bone will have a different, e.g., less dense, density, which can show up in x-ray image 530 as a lighter grayscale intensity, represented by shading 534B on bone density scale 532.

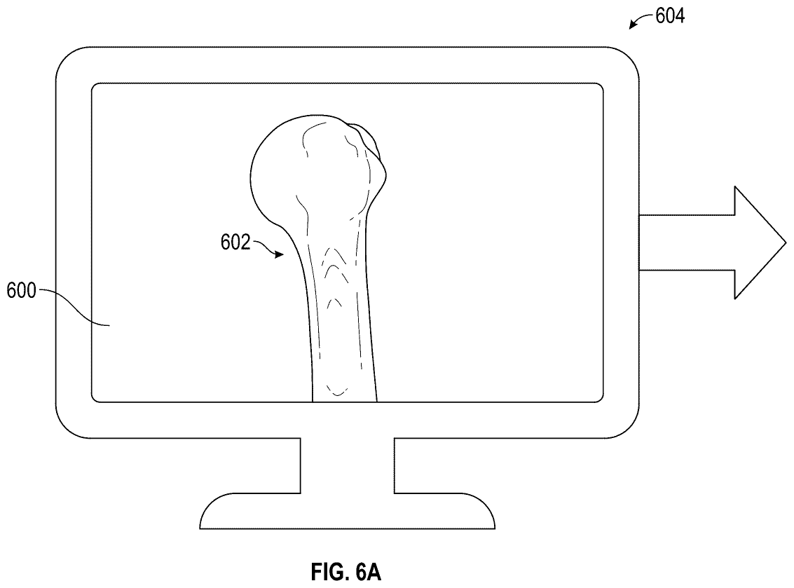

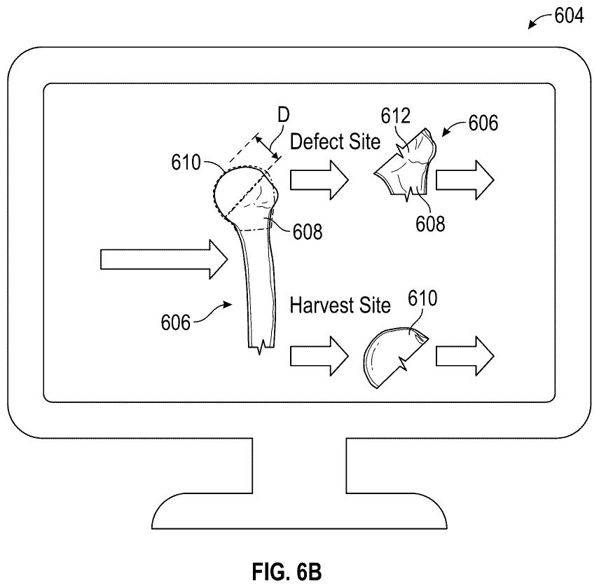

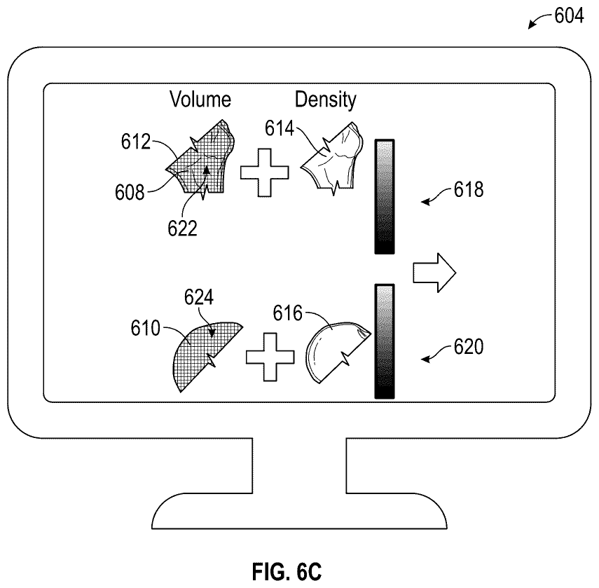

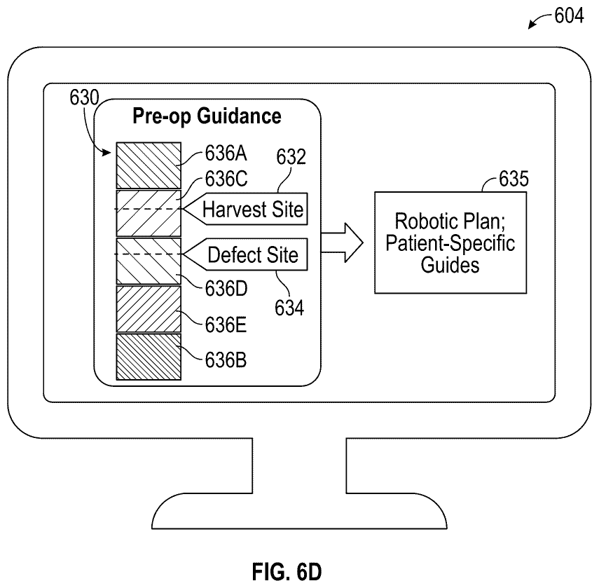

As is discussed in greater detail below, particularly with respect to FIGS. 6A-9, imaging of shoulder joint 500 can be used to generate patient-specific, three-dimensional models that can include numerical modeling including bone density and strength information. In examples, volumes of interest of bone matter that are to receive a prosthetic implant can be evaluated to determine if the volumes of interest have sufficient bone density and strength to receive and support a medical device, such as a prosthetic implant or a bone plate. In additional examples, volumes of interest of fractured bone matter that are to be repaired by reassembly of bone fragments can be evaluated to determine if the remaining bone (e.g., the portion of the bone that is still intact) has sufficient bone density and strength to support the anatomy or if the non-fractured bone is to be supplemented with a bone graft, intramedullary nail or bone plate. Additionally, volumes of interest of harvest sites for bone graft material can be analyzed to determine if volumes of bone matter having sufficient bone density and strength are available at a desired or selected harvest site.

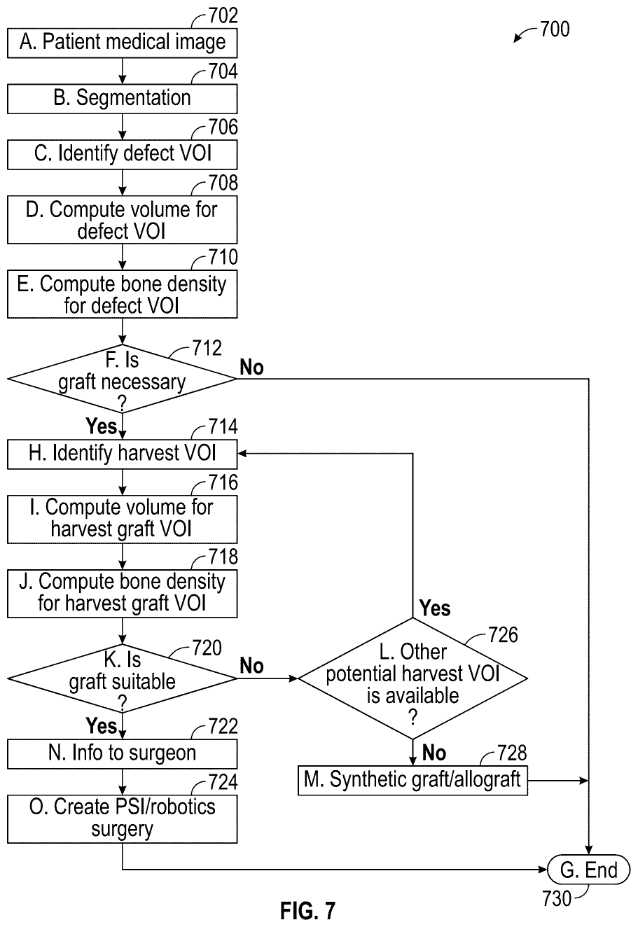

FIGS. 6A-6D illustrate operations in evaluating a bone for a bone graft. In particular, FIGS. 6A-6D illustrate operations in evaluating bone density in cancellous bone in order to determine the need or desire for a bone graft and to determine the suitability of cancellous bone for the donor bone graft from a harvest site. FIGS. 6A-6D represent examples of operations that can be used and more or fewer steps can be executed or the operations described can be performed in other sequences. FIGS. 6A-6D illustrate examples of operations that can form part of method 700 described with reference to FIG. 7. Thus, a brief overview of FIGS. 6A-6D is provided below and a more detailed description is provided with reference to FIG. 7.

FIG. 6A shows x-ray image 600 of humeral bone 602 displayed on display monitor 604. A plurality of images of humeral bone 602 from different angles can be obtained. A sufficient number of images can be obtained to determine three-dimensional bone model, as explained with reference to FIG. 6B. In examples, a CT scan of patient 110 (FIG. 1) can be used to segment humeral bone 602 by extracting the bony outline of humeral bone 602 on each scan image so that the views can be pieced together to obtain a three-dimensional model with three-dimensional bone density information. In examples, medical imaging such as CT, x-ray, MRI imaging, Dual-energy X-ray absorptiometry (DEXA) and the like can be depicted on display monitor 604 for viewing and analysis.

FIG. 6B shows bone model 606 of humeral bone 602 of FIG. 6A. As mentioned, the outline of humeral bone 602 from various images can be connected to reconstruct the 3D bone geometry of humeral bone 602 using segmentation. A 3D bone volume can also be extrapolated from 2D medical imaging using, for example, 2D to 3D methods and algorithms.

FIG. 6B shows defect site model 608 and harvest site model 610 of bone model 606. Defect site model 608 and harvest site model 610 can be added to bone model 606 via a surgeon or surgical planner. Defect site model 608 and harvest site model 610 illustrate a particular case where the defect and harvest sites are on the same bone, but defect site model 608 and harvest site model 610 can be on separate bones. Graphical user interface tools can be used to identify volumes of interest (VOIs) on bone model that form defect site model 608 and harvest site model 610. Cut surface 612 can be selected by the surgeon and can comprise a resection surface that delineates between defect site model 608 and harvest site model 610. The depth D of the cut relative to the apex of the humeral head can be selected by a surgeon or by computing system 140 and thus can vary in different scenarios. In examples, the surgical planning tools of the present disclosure can provide feedback for the placement of cut surface 612 based on bone density information and prosthetic fixation feature sizes to, for example, influence the size of bone graft used or facilitate the determination of a type of prosthesis to use.

In the example of FIG. 6B, the bone defect site can comprise a humeral head, wherein a proximal portion of the humeral head can be damaged or diseased so as to impact the ability of the humeral head to interact with a glenoid. For example, the outer cortical bone of the humeral head can be damaged. Thus, the proximal portion of the humeral head can be removed so that a prosthetic humeral head can be affixed to the humeral bone. Thus, defect site model 608 can be analyzed as described herein for the need or desirability of a bone graft. Additionally, harvest site model 610 can be analyzed for suitability as the location for bone graft material. In particular, cancellous and cortical bone material within the resected humeral head can potentially be used as bone graft material for packing into cut surface 612 of defect site model 608 to facilitate adhesion of a prosthetic fixation feature.

FIG. 6C shows defect site model 608 and harvest site model 610 of bone model 606 integrated with bone density information. Specifically, defect site density model 614 and harvest site density model 616 can be infused with density information obtained from x-ray image 600 to generate defect site model 608 and harvest site model 610, respectively, as discussed with reference to FIG. 5A and FIG. 5B. Density scale 618 and density scale 620 can be positioned adjacent defect site density model 614 and harvest site density model 616 for visualization.

FIG. 6C further shows defect site model 608 and harvest site model 610 of bone model 606 discretized into mesh 622 and mesh 624, respectively, of discretized VOI. Bone model 606 can be discretized into a mesh of elements each comprising an element and nodes, numerical value or three-dimensional (x, y, z) coordinate. The elements can consist of the original 3D pixels of the volume, and can be obtained from a meshing step of the 3D volume. The discretization elements can comprise inputs to variables within mathematical formula representing humeral bone 602. The 3D bone density can be extracted from the grayscale intensity of each pixel, for each scan image, such as x-ray image 600. Each pixel of mesh 622 and mesh 624 can be assigned a coordinate (x, y, z) in three-dimensional space relative to a common origin. The bone density in the trabecular bone VOI of the defect site can be computed using Hounsfield units or BMD for each 3D pixel or voxel, the sum/average/mean/etc. of which can provide an overall bone quality metric (such as average, mean, etc.) for the defect VOI.

FIG. 6D is a schematic view of display monitor 604 providing output indicia for evaluating bone graft site suitability. In examples, display monitor 604 can be configured to output indicia comprising bone graft feedback scale 630. Bone graft feedback scale 630 can include harvest site indicator 632 and defect site indicator 634 that can provide the surgeon or surgical planner with an overall assessment of bone density at defect site model 608 and harvest site model 610 in order to determine if a bone graft is needed and if the harvest site could provide adequate bone graft material. Placement of harvest site indicator 632 and defect site indicator 634 can be automatically determined by computing system 140 by numerically determining the density of bone material in defect site model 608 and harvest site model 610. Bone graft feedback scale 630 can reduce or eliminate the need for a person to evaluate or decode the three-dimensional modelling data.

Bone graft feedback scale 630 can range from a first color 636A that can indicate highest quality or highest density bone material to a second color 636B that can indicate lowest quality or lowest density bone material. Color 636C, color 636D and color 636E can represent bone density levels between those of first color 636A and second color 636B. These bone density values or ranges of values representing each color 636A to color 636B can be determined, for example, from indexes of bone densities, articles having published bone density guidelines, biomechanical testing, or artificial intelligence models that can determine appropriate bone density levels for general patients or specific patients. The bone graft feedback scale 630 can be completed by the harvest site indicator 632 and defect site indicator 634. The defect site indicator 634 can be help the surgeon decided if the implantation site needs grafting (e.g., if defect site indicator 634 is at color 636B), while the harvest site indicator 632 can help the surgeon decide whether his potential harvest site provide adequate bone quality for the graft (e.g., if harvest site indicator 632 is at color 636A). In principle, multiple harvest sites could be considered by the surgeon, which would involve multiple harvest site indicators.

In examples, bone graft feedback scale 630 can be updated in real-time using graphical user interfaces. For example, harvest site model 610 can be updated by moving resection plane 612 around on bone model 606 and harvest site indicator 632 can adjust accordingly (e.g., colors and position, respectively) in real-time. As such, multiple bone graft harvest sizes can be analyzed in order to find a suitable harvest site. In examples, it may also be that a harvest site with a density lower than the defect site can still be suitable for a bone graft because the bone matter can be compacted, which is where the volume calculations described with reference to FIG. 7D and FIG. 7I can be used.

A surgeon can utilize output of the surgical planning system of computing system 140 to generate surgical plan 635. Surgical plan 635 can include A) whether or not a bone graft will be needed to perform the intended procedure and B) the location of one or more bone graft harvest sites to obtain bone graft material if a bone graft is to be used to execute the surgical plan. Surgical plan 635 can include location information and geometric dimensions for removing defective bone and removing the bone graft. Surgical plan 635 can include coordinate information for a surgical robot, e.g., robotic arm 120 of robotic surgical system 115 of FIG. 1, to move bone modification tools, e.g., saws, reamers, blades, into engagement with humeral bone 602 using various cut guides. Likewise, patient-specific cutting guides can be manufactured from information obtained from defect site model 608 and harvest site model 610 to guide manual movements of modification tools into engagement with humeral bone 602 to remove defective bone matter from the defect site and donor bone matter from the harvest site for the bone graft.

FIG. 7 is a block diagram illustrating operations of method 700 for planning and performing a surgical procedure using patient-specific bone density information to predict prosthesis fixation. Operation 702 through operation 730 represent example operations that can be used and more or fewer steps can be executed or the operations described can be performed in other sequences.

At operation 702, patient data can be collected from a patient, such as patient 110 of FIG. 1. Patient information such as, age, gender, height, weight, activity level, ethnicity, diagnoses (e.g., health conditions) and the like can be collected from the patient. This biographic patient information can be stored in a computer readable medium accessible to computing system 140 (FIG. 1).

Imaging can also be collected from the same patient, including from various two-dimensional and three-dimensional. This medical imaging can be, for example, an X-ray, an MRI, ultrasound, a CT scan, a fluoroscopy, a DEXA scan, etc. Medical images of a variety of formats can be used as an input to computing system 140 (FIG. 1) to build a three-dimensional model. Medical imaging is typically performed as standard of care for diagnostics or planning purposes to, for example, identify the implant type and size that will be required for the patient.

The image files can be stored in a computer readable medium accessible to computing system 140 (FIG. 1). A plurality of images of one or more joints and bones of a patient can be obtained. The anatomy can be imaged a plurality of times in different orthogonal directions, e.g., anterior, posterior, medial and lateral. A calibration phantom or bone density marker, as mentioned above, can be positioned within each image. One calibration phantom can be used in all of the images or a plurality of calibration phantoms each having the same construction can be used. However, different types of calibration phantoms can be used so long as the same or consistent bone mineral density information can be obtained and used in a normalized fashion. In examples, CT images are used because bone density information can be readily extrapolated from CT images, particularly when used with calibration phantoms. However, bone density may be determined from other imaging techniques, such as by using ultrasound densitometry, X-ray or MRI. The medical images can be used to produce bone model 606. Imaging modalities such as MRI and DEXA imaging can be used to obtain whole-body scans.

The graft harvest site may be near the defect site, and the medical image for the procedure planning may be used alone, since such imaging will include both the defect site and the harvest site. For example, the humeral head bone can be the harvest site for the same humeral bone, as shown in FIG. 6B. In another example, the humeral head bone can be the harvest site for a glenoid void, so that both the harvest and defect side are on different bones but are visible on a same medical image. It is also possible that the harvest site is away from the defect site, and another medical image can then be obtained. Furthermore, extra imaging can be obtained for alternative harvest sites. In a particular example, DEXA imaging can be used where the whole body is scanned and algorithms can fully automatically screen traditional graft donor sites for suitability, following the procedure described below. Thus, only one set of imaging can be obtained if the defect site and the harvest site are available in the same view. However, a second set of imaging can be obtained if the harvest site is located away from, e.g., on a different bone, the defect site. Further, three or more sets of imaging can be obtained if back-up harvesting sites are to be analyzed.

At operation 704, bone model 606 can be generated from the images obtained at operation 702. In examples, bone model 606 can be constructed by segmentation of images obtained at operation 702. Bone model 606 can be a patient-specific model of the patient data. Three-dimensional models can be produced from the two-dimensional and three-dimensional imaging previously obtained at operation 702. The three-dimensional models can represent virtual, digital models of anatomy of the patient. The patient-specific models can comprise numerical models wherein the bone matter is assigned a plurality of numerical points in 3D space that can be assigned as variables in one or more mathematical equations that represent the bone matter. The numerical points can be assigned values, such as based on the density (particularly of interest for cancellous bone analysis) of the bone matter at that three-dimensional location in the model, as can be determined from analysis of the imaging. The patient-specific model of the patient can include appropriate scaling of bones in a joint, the physical relationship of the bones in a joint and bone density information for each bone in a joint.

In examples, the three-dimensional model can comprise a mean bone model or a statistical shape and intensity (SSIM) model into which patient-specific bone density information can be imparted. The bone model or mean bone can be a generalized model based on multiple patient bone models and can be selected from a principal component analysis (“PCA”) based statistical bone atlas. One such a priori bone atlas includes a dataset of a large number of dry femur and tibia bone pairs, scanned by CT and segmented to create models of each bone. The method of building and using one such statistical atlas is described in MAHFOUZ M et al., “Automatic Methods for Characterization of Sexual Dimorphism of Adult Femora: Distal Femur,” Computer Methods in Biomechanics and Biomedical Engineering, 10 (6) 2007, the disclosure of which is incorporated herein by reference in its entirety. Further description of such a bone model and associated procedures for generating a bone model are described in Pat. No. U.S. Pat. No. 10,512,245 to Mahfouz titled “Method and apparatus for three-dimensional reconstruction of a joint using ultrasound,” the contents of which are hereby incorporated in their entirety by this reference. Additional examples of generating mean bone models are described in Pat. No. U.S. Pat. No. 10,130,478 to Mahfouz titled “Ethnic-specific orthopaedic implants and custom cutting jig,” the contents of which are hereby incorporated in their entirety by this reference. See Incorporating Population-Level Variability in Orthopedic Biomechanical Analysis: A Review by Bischoff et al., Journal of Biomechanical Engineering, February 2014, Vol. 136/021004-1, which is hereby incorporated by reference in its entirety, for further description of statistical shape and intensity models.

At operation 706, the defect volume of interest (VOI) can be identified. On bone model 606, a surgeon or surgical planner can manually identify the volume of interest (VOI) for defect site model 608 and harvest site model 610, using a dedicated application within the planning software executable on computing system 140 (FIG. 1). The surgeon or surgical planner can utilize computer interface devices on computing system 140 to draw lines or points on bone model 606 to determine a volume of interest for a defect site. The defect site can comprise a location where weak or diseased bone is to be removed and replaced with a prosthetic device. In examples, bone modification plotting can be electronically drawn directly onto bone model 606 of patient 110 using a mouse, a touch screen, or a virtual reality or augmented reality headset, such as the Microsoft HoloLens, and the like, to manipulate resection planes in a graphical user interface (e.g., a video display device). Bone model 606 can be rotated on the screen of display monitor 604 and the surgeon can draw digital lines on bone model 606 to produce the VOI. This step can also be automated using specific segmentation algorithms that isolate the trabecular bone on each 2D slice and recreate the 3D volume or Artificial Intelligence to recognize typical VOI on the image. For example, AI models can be trained to identify defect locations in imaging of bones based input from previously performed orthopedic procedures for the same or similar types of procedure.

The volume of interest can be calculated by computing system 140 based on an analysis of the lines and points drawn by the surgeon on bone model 606. The surgeon can place the bone defect VOI based on resection planes, reamed or drilled channels and the size of anchoring features, such as stem 204, blades 313 and fasteners 404. The VOI can be selected to encompass prosthetic anchoring components of one or more different implant types. Additionally, multiple VOIs for different prosthetic anchoring components can be selected as alternative plans.

At operation 708, the volume of the VOI for defect site model 608 can be determined. Computing system 140 can analyze the bone model 606 to calculate the volume for defect site model 608. Computing system 140 can utilize geometric information within bone model 606, such as points and meshes, to determine the volume. The lines and points drawn by the surgeon at operation 706 can be connected via computing system 140 to define a volume of interest.

At operation 710, bone density information can be determined from the imaging and incorporated into the patient-specific model of the patient. For example, bone density of each bone in the medical images can be determined using density markers or calibration phantom placed in the scanner. Bone volume and quality quantification can utilize calibrated medical images, as shown in FIGS. 5A and 5B. Calibration of the dimensions on the image is often part of the standard pre-operative planning to determine implant size. As such, size calibration markers, such as spheres of known sized can be included in the imaging to facilitate determination of volume. Bone quality for CT scanning can be calibrated by means of a calibration phantom that needs to be scanned with the patient or using phantomless methods that use the known density of other materials on the image (such as air, cortical bone, muscle, fat). DEXA images do not require bone quality calibration.

In examples, the native grayscale intensity of the voxels of the original images can be turned into bone density values using a calibration phantom as a reference. Such phantom contains a series of inserts made of various known densities of radio-opaque mineral material (e.g. hydroxyapatite), which allow the user or software to establish a relationship between the original grey value of a voxel and the underlying mineral density of the scanned tissue. In another example, bone density of each bone in the medical images can be determined using references already present in the image, such as air, fat, muscle etc., whose density is known. A so-called phantomless calibration can therefore be established, for instance with AI algorithms. In case of two-dimensional images, bone density information from the different orthogonal views can be pieced together during construction of three-dimensional models for each bone. In case of CT scans, piecing together of images to formulate bone density information is conducted automatically. Examples of bone density markers, calibration phantoms and phantomless calibration are described in Pat. No. U.S. Pat. No. 10,736,601 to Kopperdahl et al., titled “Quantitative phantomless calibration of computed tomography scans,” the contents of which are hereby incorporated by reference. In examples, bone density patterns can be incorporated into three-dimensional bone models to, for example, provide an indication of where a bone graft is needed or desired and where bone graft material can be harvested from. Such information can be used to pre-operatively plan procedures to determine if additional bone graft harvesting procedures will be executed.

In a first example, the bone density pattern of the specific patient can be incorporated into a three-dimensional model of that patient's bone. In a second example, the bone density pattern of the specific patient can be incorporated into a mean bone model having dimensions of a mean bone. In such examples, the specific patient's bone density information can be directly analyzed in the regions of interest, relying on the geometric information provided by the mean bone model. For example, bone density within the volumes of interest identified in operation 708 can be analyzed to determine if bone graft operations will be performed intra-operatively. In a third example, the bone density pattern of the specific patient can be compared to bone density patterns of a mean bone model having mean bone density information, where mean bone density information can be used as an estimate for the specific patient. In a fourth example, the bone density pattern of the specific patient can be compared to bone density patterns in a database of an artificial intelligence or machine-learning engine. In such examples, the specific patient's bone density information can be compared to aggregated bone density information of a fixed or changing population of patients to first identify areas of healthy and unhealthy bone before simulating the implant of a prosthesis. Such comparisons to bone density information of a population can expedite or speed up the simulation process to initially eliminate untenable prosthesis selection and placement before running simulations.

In examples, defect site density model 614 and harvest site density model 616 can be constructed using osteoabsorptiometry as is described in von Eisenhart-Rothe R, Müller-Gerbl M, Wiedemann E, Englmeier K H, Graichen H. Functional malcentering of the humeral head and asymmetric long-term stress on the glenoid: potential reasons for glenoid loosening in total shoulder arthroplasty. J Shoulder Elbow Surg. 2008 Sep.-Oct.; 17 (5): 695-702. doi: 10.1016/j.jse.2008.02.008. Epub 2008 Jun. 16. PMID: 18558500, which is hereby incorporated by this reference in its entirety.

In examples, defect site density model 614 and harvest site density model 616 can be constructed using CT scans as is described in Couteau B, Mansat P, Mansat M, Darmana R, Egan J. In vivo characterization of glenoid with use of computed tomography. J Shoulder Elbow Surg. 2001 Mar.-Apr.; 10 (2): 116-22. doi: 10.1067/mse.2001.112884. PMID: 11307073, which is hereby incorporated by this reference in its entirety.

At operation 712, a surgeon or computing system 140 (FIG. 1) can determine if a bone graft is necessary. For example, the density of cancellous bone matter within the volume of interest for the defect site can be analyzed. Threshold methods for bone density can be used to identify the VOI where trabecular bone of insufficient density lies, and which will require grafting. For example, bone graft feedback scale 630 (FIG. 6D) can be utilized to determine if the density of bone in the volume of interest of the defect sight is sufficiently dense to allow a bone anchor to attach thereto and support the bone. Bone graft feedback scale 630 can be used to provide an indication of the defect site bone density relative to average and threshold values. The need for a bone graft can be confirmed by the tool, by providing a density assessment of the defect region. For example, the bone density at defect site model 608 can be compared to values from the literature, or from medical imaging databases. If the density at defect site model 608 is equivalent to average values, the graft may not be necessary, and the algorithm can stop.

If the surgeon determines that the bone density is sufficiently high, method 700 can continue to operation 730 to end the pre-operative planning. In such case, a bone graft from a harvest site is not needed. Thus, a surgical plan can be prepared wherein the prosthetic implant can be directly implanted into native bone at the defect site.

If the surgeon determines that the bone density is too low, method 700 can continue to operation 714. For example, if the surgeon or computing system 140 determines that the bone density is insufficiently dense to provide adequate support for a prosthesis, it can be determined to use a bone graft material having higher bone density to provide a scaffold for the osseointegration of the prosthesis. Bone grafts act as a mineral reservoir which induces new bone formation.

At operation 714, a volume of interest on the bone model can be determined for the harvest site. A surgeon or surgical planner can utilize computer interface devices to identify the harvest site VOI on bone model 606, as described with reference to operation 706. If the bone graft is necessary, as determined at operation 712, one or more graft harvest site can be identified. Here the surgeon may directly image another potential graft site based on experience of the generally adequate harvest sites, or the algorithm may directly propose an adequate harvest location based on the analysis of the defect site. Alternatively, the surgeon may take another image at another location at another time. The bone volume and density can then be evaluated for the harvest graft VOI, following the same method as for the defect site.

At operation 716, the volume of the harvest site VOI can be determined, similarly described with reference to operation 708. Computing system 140 (FIG. 1) can also be used to evaluate if the volume of the VOI for the harvest site is sufficiently large to cover or fill the VOI for the defect site.

At operation 718, bone density for the VOI of the harvest site can be determined. Similar methods and procedures as used at operation 710 can be used at operation 716. In examples, a graphical representation of the harvest site VOI can be placed in juxtaposition next to a graphical representation of the defect site VOI to allow for visual confirmation that the harvest site will provide an adequately large volume.

At operation 720, a surgeon or a surgical planner can determine if the bone graft evaluated at operation 714 to operation 718 is suitable for use to fill the VOI of the defect site. Harvest site model 610 can be evaluated for suitability. For example, a target density can be set for harvest site model 610 based on the literature or from medical imaging databases. Also, the density near the defect site, or the density on another portion of humeral bone 602 (if applicable) may be used as a target. If the VOI in the harvesting site does not reach that target, it may be considered insufficient. More complex evaluation methods could estimate the total available bone mass by multiplying the volume by the density of each voxel in the VOI and adding the value for each pixel to evaluate the graft mass. This allows considering a small volume of high-density bone that may be as suitable as a large volume of low-density bone. Similarly, there may also be the possibility to crush bone, which can include identifying a larger volume harvest site than what is needed, and predict the effective density when consolidated down to the target volume. The solution then is not a solid piece of trabecular bone, but rather a volume of bone paste from either cortical or trabecular, or a combination thereof, sufficient to fill the VOI. Another method could consist in using AI methods, trained on surgeon decisions made for use cases as the tool is being used and data is collected.

Artificial intelligence and machine learning can be utilized to inform the simulations, such as by training the software to recognize areas of weak bone density and plan appropriate bone graft selection and bone modifications, e.g., resections, that avoid obtaining the bone grafts from weak bone and weakening the bone. For example, bone density information from the patient population used to generate various patient-specific models of various patients over time can be used to identify known areas of healthy and weak bone density and what associated healthy and weak bone areas look like in imaging. Computing system 140 can then review the patient-specific model of a particular patient to identify similar patterns in the imaging that resemble the healthy and weak bone areas to thereby find a prosthesis having fixation features that will engage as much healthy bone as possible or as desired to meet certain risk criterion. Thus, a patient-specific bone density pattern can be compared to a library of bone density patterns or amalgamations of bone density patterns to help identify areas of weak, low-density bone or areas of strong, high-density bone. The patient-specific bone density pattern can then be incorporated into the bone density library and amalgamations for future use with other patients.

If the surgeon determines that the bone density is sufficiently high at the harvest site and there is a large enough volume for the bone graft needed at the defect site, method 700 can continue to operation 722, discussed below, so the surgeon can receive and approve a surgical plan involving the bone graft.

If the surgeon determines that the bone density is too low, method 700 can continue to operation 726, discussed below, to find alternative harvest sites or use a synthetic or heterologous bone graft.

At operation 722, information relating to the defect site and harvest site can be provided to the surgeon. The surgical plan can include the location and volume of bone matter to be removed from the defect site and the location and volume of bone matter for the bone graft. For example, a paper copy a surgical plan can be provided to the surgeon. Additionally, an electronic copy of the surgical plan can be displayed on a video monitor or stored in computer memory. The surgical plan can comprise images of the bone model including the densities and density scale 618 and density scale 620, as well as bone graft feedback scale 630, as shown in FIGS. 6C and 6D. The surgeon can approve the plan for incorporation into the overall surgical plan for the orthopedic or trauma surgery.

At operation 724, specifications for patient-specific instruments and positioning instructions for robotic surgery can be prepared. Operation 724 can be optional if patient-specific instruments and robot guided surgery are not to be used in executing the overall surgical plan. If desired, an exact bone donor contour can be defined, and surgical aids can be implemented to harvest the required bone. This can ensure that no more bone than required is harvested, thereby limiting morbidity at the harvest site, and no less bone than required is harvested, thereby limiting the risk of graft failure. These surgical aids include, for example, personalized surgical instruments and guides or robotics surgery instrumentation and coordinates. Further technologies may be used to facilitate the grafting procedure, such as virtual reality to help best position the cortical bone graft in the defect.

The patient-specific instruments and any associated patient-specific implants and bone grafts can be generally designed and manufactured based on computer modeling of the patient's 3-D anatomic image generated from medical image scans including, for example, X-rays, MRI, CT, PET, ultrasound or other medical scans. The patient-specific instruments can have a three-dimensional engagement surface that is complementary and made to substantially mate and match in only one position (i.e., as a substantially negative or mirror or inverse surface) with a three-dimensional bone surface with or without associated soft tissues, which is reconstructed as a 3-D image via the aforementioned CAD or software. Very small irregularities need not be incorporated in the three-dimensional engagement surface. The patient-specific instruments can include custom-made guiding formations, such as, for example, guiding bores or cannulated guiding posts or cannulated guiding extensions or receptacles that can be used for supporting or guiding other instruments, such as drill guides, reamers, cutters, cutting guides and cutting blocks or for inserting guiding pins, K-wire, or other fasteners according to a surgeon-approved pre-operative plan.

Robot guidance instructions can be prepared for robotic surgical systems to make incisions or place instruments relative to defect site model 608 and harvest site model 610. The guidance information can include three-dimensional coordinates for robotic arm 120 to position appropriate bone modification tools adjacent humeral bone 602 to remove the harvest VOI without removing unacceptable levels of additional bone. Thus, cutting instruments can be guided into the bone matter to minimize trauma and avoid harvesting excess quantities of bone matter.

At operation 726, one or more potential harvest sites can be analyzed for suitable bone densities if the volume or density from the donor site is insufficient. For each potential harvest site, operation 714 to operation 720 can be repeated until a suitable harvest site can be found and decided upon by the surgeon. Suitable harvest sites can have acceptably high levels of bone density and sufficient volume to fill the defect VOI. As discussed at operation 728, a surgeon or surgical planner can consider another graft material such as synthetic material, or an allograft. Thus, bone graft material can be combined from multiple sites or alternative harvest sites can be considered until one of sufficient size is found to avoid making multiple harvest sites on the patient.

If a suitable back-up harvest site is found, method 700 can continue to operation 724 and operation 730.

If a suitable back-up harvest site cannot be found, method 700 can continue to operation 728.

At operation 728, a surgeon can decide if a synthetic graft or an allograft can be used at the defect site rather than an autograft. Synthetic graft material and allograft material can be used with or alternatively to autograft material.

At operation 730, the evaluation and planning process can be completed. The surgical plan for performing the bone graft procedure can be incorporated into the overall surgical plan for performing the prosthesis implantation.

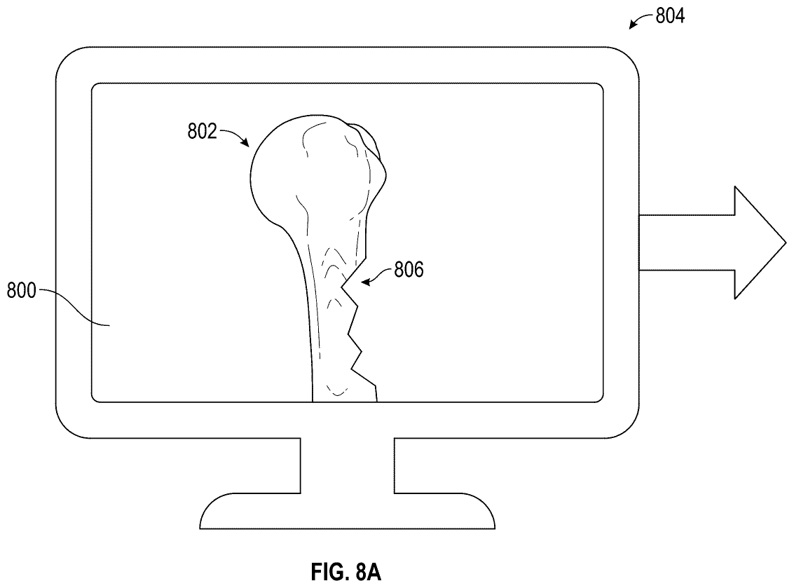

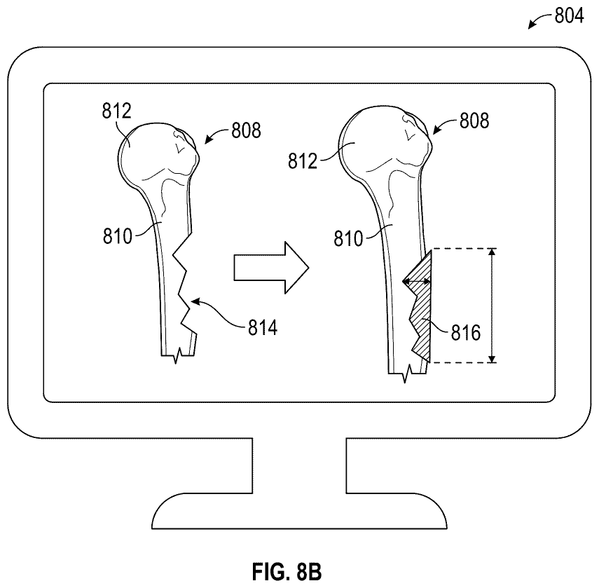

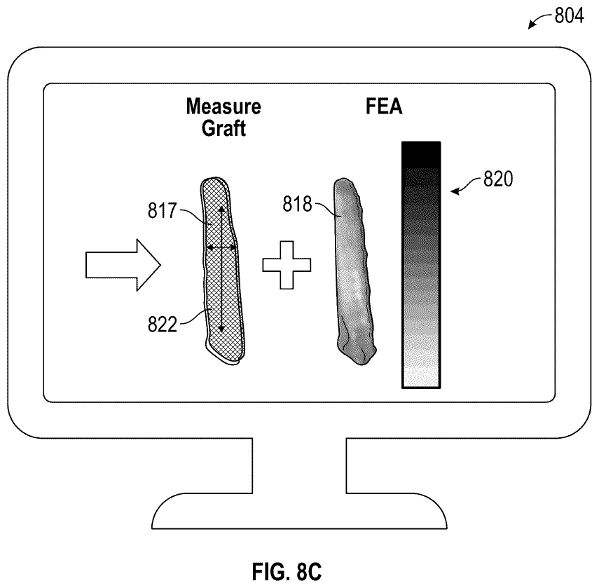

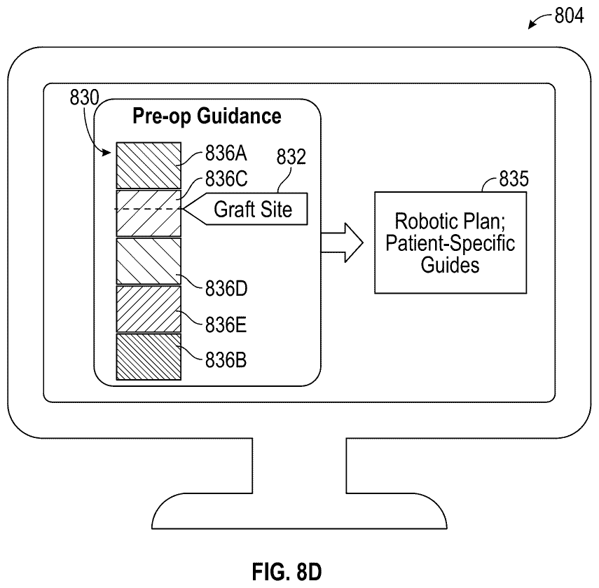

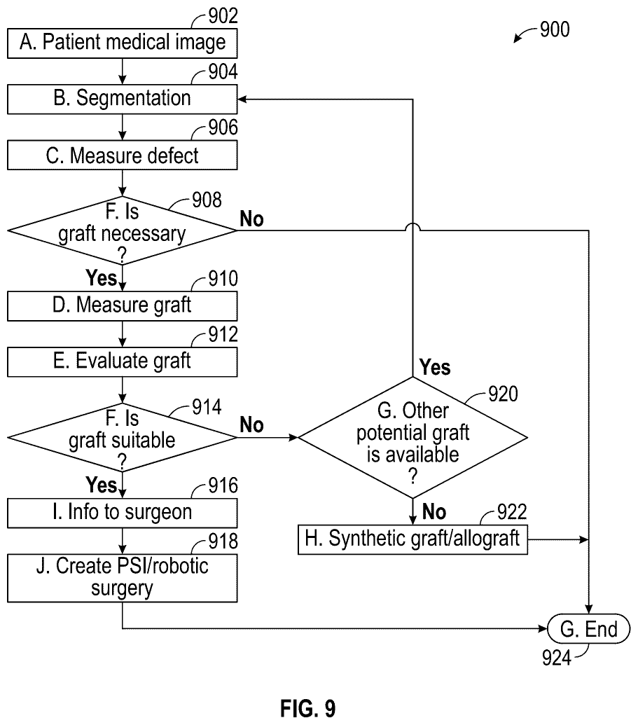

FIGS. 8A-8D illustrate operations in evaluating a bone for a cortical bone graft. In particular, FIGS. 8A-8D illustrate operations in evaluating bone strength in cortical bone in order to determine the need or desire for a bone graft and to determine the suitability of cortical bone for the donor bone graft from a harvest site. FIGS. 8A-8D represent examples of operations that can be used and more or fewer steps can be executed or the operations described can be performed in other sequences. FIGS. 8A-8D illustrate examples of operations that can form part of method 900 described with reference to FIG. 9. Thus, a brief overview of FIGS. 8A-8D is provided below and a more detailed description is provided with reference to FIG. 9.

FIG. 8A shows x-ray image 800 of humeral bone 802 displayed on display monitor 804. Humeral bone 802 can include defect 806. Imaging of humeral bone 802 can be obtained as described with reference to FIG. 6A. Further, defect 806 can be included in the imaging so that the geometry, e.g., the lengths of various edges, surface area of various surfaces and angles between surfaces, and volume of defect 806 can be determined.

FIG. 8B shows bone model 808 of humeral bone 802 of FIG. 8A. Bone model 808 can comprise shaft model 810, head model 812 and defect model 814. Furthermore, bone graft VOI model 816 is also shown. Bone model 808 can be constructed in a similar fashion to bone model 606 as described with reference to FIG. 6B. Bone graft VOI model 816 can have an inverse shape of defect model 814. Bone graft VOI model 816 can comprise a volume of bone matter having a shape and size configured to fit into and fill defect 806. Bone graft VOI model 816 can be used to help identify volumes of bone matter at harvest sites that are sufficiently large to fill defect 806.

In the example of FIG. 8B, the bone defect site can comprise a humeral shaft where a portion of fragmented bone has been removed. Humeral bone 802 can become damaged from an inflicted trauma, which can result in small bone fragments being formed in defect 806. The bone fragments can primarily be formed of cortical bone. If the bone fragments are too small to be reconstructed, a bone graft can be used to fill defect 806. In examples, defect 806 could be left unfilled and humeral bone 802 can be augmented with an intramedullary nail or a bone plate. Finite Element Analysis (FEA), as described herein, can be utilized to analyze the strength, e.g., compressive strength in Pascals (N/m²), of humeral bone 802, e.g., bone model 808, and determine if humeral bone 802 provides sufficient structural support with defect 806. However, it is frequently desirable to fill defect 806 in some manner. As such, it can be desirable to harvest donor bone material from another location on the patient. In examples, donor bones for long bones, such as humerus bones and femoral bones, can comprise rib bones, or the fibula. Thus, defect 806 and defect model 814 can be analyzed, such as by using FEA as described herein, for suitability of a bone graft to provide adequate structural support to humeral bone 802. For example, the size and shape of rib bone can be compared to the size and shape of defect model 814 to determine suitability as a bone graft.

FIG. 8C shows bone graft model 817, such as can be taken from a donor bone, such as a rib. Specifically, bone graft model 817 can be integrated with finite element analysis (FEA) analysis to produce FEA model 818. FEA output scale (such as stress/strain/deformation, etc) scale 820 can be positioned adjacent FEA model 818 for visualization. FIG. 8C further shows bone graft model 817 discretized into mesh 822 of finite elements. Bone model 808, as well as bone graft model 817, can be discretized into finite elements each comprising an element and nodes, numerical value or three-dimensional (x, y, z) coordinate. The finite elements can comprise inputs to variables within mathematical formula representing bone model 808.

Bone graft model 817 can comprise a model of a rib bone of the patient of humeral bone 802. A surgeon or surgical planner can digitally position bone graft model 817 proximate defect model 814 or bone graft VOI model 816 to determine suitable size. Additionally, computing system 140 can calculate the outer dimensions of defect model 814 to determine if bone graft model 817 will encompass defect model 814. Finite element analysis can be performed on bone model 808 with bone graft model 817 filled in defect model 814 to determine if bone graft model 817 will provide adequate structural stability to humeral bone 802. In addition to the FEA strength evaluation, bone remodeling algorithms could be used in combination with the FEA model to determine the longer-term graft performance.

FIG. 8D is a schematic view of display monitor 804 providing output indicia for evaluating bone graft site suitability. In examples, display monitor 804 can be configured to output indicia comprising bone graft feedback scale 830. Bone graft feedback scale 830 can include graft site indicator 832 that can provide the surgeon or surgical planner with an overall assessment of bone strength at FEA model 818 in order to determine if a bone graft is needed and if the harvest site could provide adequate bone graft material. In another example, bone graft feedback scales 830 and graft site indicator 832 can provide feedback based on geometric shape alone, e.g., size, of the harvest site, such as to provide feedback as to whether or not the harvest site is large enough for the defect site, without relying on FEA. Placement of graft site indicator 832 can be automatically determined by computing system 140 by numerically determining the strength of FEA model 818. Bone graft feedback scale 830 can reduce or eliminate the need for a person to evaluate or decode the simulation data.

Bone graft feedback scale 830 can have a first color 836A that can indicate high strength of bone model 808 and a high or acceptable potential for a bone graft site and second color 836B can indicate low strength of bone model 808 and a low or unacceptable potential for a bone graft site. Color 836C, color 836D and color 836E can represent bone strength levels between those of first color 836A and second color 836B. Bone strength values or ranges of values representing each of color 836C to color 836E can be determined from indexes of bone strengths, articles having published bone strength guidelines or artificial intelligence models that can determine appropriate bone strength levels for general patients or specific patients. If graft site indicator 832 is at first color 836A, a surgeon can determine to use the bone graft obtained from the harvest site, e.g., the rib cage. If graft site indicator 832 is at second color 636B, the surgeon can determine to not use the bone graft obtained from the harvest site, and can look for another bone graft site, such as by using a larger rib bone. If graft site indicator 832 is at other colors, the surgeon can utilize judgement and experience to determine if a bone graft is needed and if the harvest site will provide an adequate bone graft.

In examples, bone graft feedback scale 830 can be updated in real-time using graphical user interfaces. For example, the position of FEA model 818 can be modified on bone model 808 and graft site indicator 832 can adjust accordingly (e.g., colors and position, respectively) in real-time, additionally using AI trained on multiple FE analyses in various examples. As such, multiple bone graft harvest sites can be analyzed in order to find a suitable harvest site.

A surgeon can utilize output of the surgical planning system of computing system 140 to generate surgical plan 835. Surgical plan 835 can include A) whether or not a bone graft will be needed to perform the intended procedure and B) the location of one or more bone graft harvest sites to obtain bone graft material if a bone graft is to be used to execute the surgical plan. Surgical plan 835 can include location information and geometric dimensions for filling a defective bone and removing the bone graft. Surgical plan 835 can include coordinate information for a surgical robot, e.g., robotic arm 120 of robotic surgical system 115 of FIG. 1, to move bone modification tools, e.g., saws, reamers, blades, into engagement with humeral bone 802 using various cut guides. Likewise, patient-specific cutting guides can be manufactured from information obtained from bone graft VOI model 816 to guide manual movements of modification tools into engagement with humeral bone 802 to remove defective bone matter from the defect site and donor bone matter from the harvest site for the bone graft.

The systems, devices and methods of the present disclosure can evaluate bone matter proximate a defect site, e.g., where a bone fracture has occurred or where a bone has been broken into small pieces, to determine if the remaining intact bone matter would require a bone graft to facilitate proper healing or to allow for proper attachment of a bone plate, or if a bone plate or intramedullary nail is needed or not needed. In examples, the systems, devices and methods of the present disclosure can facilitate the use of bone plates by allowing bone pieces of sufficiently large size and density to engage with fasteners 404.

FIG. 9 is a block diagram illustrating operations of method 900 for evaluating and performing a bone graft procedure for cortical bone matter. Operation 902 through operation 924 represent example operations that can be used and more or fewer steps can be executed or the operations described can be performed in other sequences.

In a cortical bone graft, the density is less relevant, because the cortical bone density varies marginally between anatomical regions. A goal of a cortical bone graft is to provide structural support by filling a gap, in a case of fracture for example, therefore the shape of the graft is more important. The bone graft should be of sufficient size to fill the gap and appropriately transmit forces, such as to other intact portions of the bone.

At operation 902, patient data can be collected from a patient, such as patient 110 of FIG. 1. Patient data can be collected as described with reference to operation 702. Imaging of both the defect site of defect model 814 and the harvest bone (such as a rib, a fibula, etc.) for bone graft model 817 can be obtained. As discussed, medical imaging of a graft harvest site is useful in assessing a cortical bone graft. The graft harvest site may be near the defect site, and the medical image for the procedure planning may be used alone. It is also possible that the harvest site is away from the defect site, and another medical image is then necessary. Here the surgeon may directly image another potential graft site based on experience of the generally adequate harvest sites, or the algorithm of computing system 140 can directly propose an adequate harvest location based on the analysis of the defect site, such as by using artificial intelligence models or database information. An additional image of the donor site may be necessary if this anatomical region is not in the field of view of the first image. Medical imaging is very often performed as standard of care for planning purposes in cases requiring a cortical graft (e.g. fracture, reconstructive surgery). This medical imaging can be, for example, an X-ray, a 3D x-ray, an MRI, a CT scan, a fluoroscopy, a DEXA scan, etc.

At operation 904, 3D bone models of the defect and the graft are created using segmentation techniques. The 3D bone volume may also be extrapolated from a 2D image using, for example, 2D to 3D methods. The three-dimensional bone models can be constructed as described with reference to operation 704.

At operation 906, the defect volume of interest (VOI) can be identified. On bone model 808, the surgeon or a surgical planner can manually measure the size of defect model 814 using a dedicated application within the planning software of computing system 140. The measurements can be computed using manual methods (e.g. the user can paint the volume on bone model 808, use Boolean operations to virtually cut the bone, measure distances, etc.). Finite element analysis (FEA) can be added to assess the strength of the bone with the defect. Bone remodeling algorithms may also be used in combination with finite element model to determine the longer-term graft performance.

At operation 908, a surgeon can analyze the defect site to determine if a bone graft is necessary. For example, bone model 808 having defect model 814 can be analyzed to determine if the intact bone matter of bone model 808 is sufficient to allow humeral bone 802 to function adequately. If the size and/or strength is sufficient, no graft is needed and the procedure can be ended at operation 924. In such cases, a bone plate or an intramedullary nail can be used. If the strength of the bone is insufficient and/or the size of the defect too large, it can be decided to use a bone graft, with or without a plate or nail, and the procedure can continue to operation 910.

At operation 910, if grafting is necessary, the surgeon or a surgical planner can manually, e.g., by manipulating interface devices of computing system 140, measure the size of the bone graft. For example, bone graft VOI model 816 can be measured for comparison to bone graft model 817, using the same method as described above for the defect at operation 906. Computing system 140 can perform calculations automatically based on surgeon input, e.g., drawing on bone model 808, to determine the size of the bone graft.

At operation 912, bone graft model 817 can be evaluated geometrically or mechanically. The volume of bone graft model 817 can be compared to the volume of bone graft VOI model 816 to determine if bone graft model 817 will provide coverage to defect 806. For example, automated methods may be implemented, by overlaying the two bone volumes and maximizing fit of the graft volume of the defect volume and ensure geometric fit of the graft at the defect site. FEA can be used to mechanically evaluate if the graft will be sufficient to support the expected load.

At operation 914, the bone graft can be evaluated for suitability using results from operation 912. Simulations can be run based on the patient-specific model using biomechanical data and/or patient data. The simulations can comprise a plurality of different scenarios, which can include: 1) simulated prosthesis (e.g., bone plate) and bone graft if desired to be used, 2) a simulated fracture plane as well as any other cuts or bone alterations due to the selected prosthesis or due to defect 806 and a bone graft, 3), a simulated positioning of the selected prosthesis and graft at the defect location, and 4) a simulated post-operative usage (loading) of humeral bone 802. The simulations can involve analyzing the bone matter of the native bone and of the bone graft to determine, for example, how well the bone matter holds up under loading from the body of the patient and movement of the body of the patient.

Simulations can be run on the patient-specific model of the patient by solving equations related to the finite element analysis. The model can apply force data and material data (e.g., bone density) and simulate the biomechanical behavior of the system. As such, loading on various surfaces and portions of the bones in a joint can be simulated to determine the risk level for various factors, such as bone or implant breakage and implant loosening.

In examples, stability of an implanted prosthesis can be determined as is described in Favre P, Seebeck J, Thistlethwaite P A, Obrist M, Steffens J G, Hopkins A R, Hulme P A. In vitro initial stability of a stemless humeral implant. Clin Biomech (Bristol, Avon). 2016 February; 32:113-7. doi: 10.1016/j.clinbiomech.2015.12.004. Epub 2015 Dec. 21. PMID: 26747397, the contents of which are hereby incorporated by reference.

The simulations can solve equations to determine, for example, the stress induced in bone graft VOI model 816 during movements and loading of humeral bone 802. Finite element simulations can quantify the loading on bone graft VOI model 816 when the patient is performing an activity and compare this loading against maximum thresholds to evaluate the likelihood that the bone graft will provide adequate support to humeral bone 802, either directly or over time. The simulations can additionally solve mechanics of materials equations to determine, for example, the strength of bone where modifications or bone grafting has occurred to determine the likelihood of the bone cracking or breaking during the implantation procedure or post-operatively during use by the patient. Finally, bone remodeling algorithms could be coupled with the FEA to determine the longer-term graft performance.

At operation 916, if the bone graft is suitable, the information can be passed on to the surgeon to approve the plan. Further information that can be provided by the FEA simulation can include an analysis of different loading scenarios to help provide guidance on which activities can be performed during the rehabilitation period, or if a given plate/nail/cerclage system may be necessary to stabilize the graft. If a plating system was deemed necessary, the FEA could help determine the needed size, type and position of the plate/nail/cerclage to best stabilize the bone.

The surgical plan can include the location and volume of bone matter to be removed from the defect site, if performed, and the location and volume of bone matter for the bone graft. For example, a paper copy a surgical plan can be provided to the surgeon. Additionally, an electronic copy of the surgical plan can be displayed on a video monitor or stored in computer memory. The surgical plan can comprise images of the bone model 808 and bone graft model 817 including FEA output (e.g. stress, strain, deformation, etc.) scale 820 and bone graft feedback scale 830, as shown in FIGS. 8C and 8D. The surgeon can approve the plan for incorporation into the overall surgical plan for the orthopedic or trauma surgery.

At operation 918, if deemed desirable, an exact bone donor graft contour can be defined, and surgical aids can be implemented to harvest the required bone that will optimally fill the gap. This ensures no more bone than required is harvested, thereby limiting morbidity at the harvest site, and no less bone than required is harvested, thereby limiting the risk of graft failure. These aids include, for example, personalized surgical instruments and guides or robotics surgery. Further technologies may be used to facilitate the grafting procedure, such as virtual reality to help best position the cortical bone graft in the defect. Patient-specific instrumentation and robotic guidance information can be generated as described at operation 724.

At operation 920, if the bone graft is insufficient, the surgeon or surgical planner may consider another donor site. One or more potential harvest sites can be analyzed for suitable bone densities if the volume or density from the donor site is insufficient. For each potential harvest site, operation 904 to operation 914 can be repeated until a suitable harvest site can be found and decided upon by the surgeon. Suitable harvest sites can have acceptably high levels of bone strength and sufficient volume to fill the defect VOI. As discussed at operation 922, a surgeon or surgical planner can consider another graft material such as synthetic material, or an allograft. Thus, bone graft material can be combined from multiple sites or alternative harvest sites can be considered until one of sufficient size is found to avoid making multiple harvest sites on the patient.

At operation 922, surgeon can decide if a synthetic graft or an allograft can be used at the defect site rather than an autograft. Synthetic graft material and allograft material can be used with or alternatively to autograft material.

At operation 924, the evaluation and planning process can be completed. The surgical plan for performing the bone graft procedure can be incorporated into the overall surgical plan for performing the prosthesis implantation.

FIG. 10 illustrates system 1000 for performing techniques described herein, in accordance with some embodiments. System 1000 is an example of a system that can incorporate surgical system 100 of FIG. 1. System 1000 can include planning system 1002, surgical system 1004, display device 1006 and control system 1008. Planning system 1002 can comprise simulation engine 1010 and artificial intelligence engine 1012. Surgical system 1004 can comprise tracking system 1014 and robot system 1016. Display device 1006 can comprise graphical user interface 1018. Control system 1008 can comprise processor 1020 and memory 1022.

Tracking system 1014 can comprise tracking system 165 described with reference to FIG. 1 and can comprise tracking elements 170, cameras, registration devices and the like.

Robot system 1016 can comprise the robotic system 115 described with reference to FIG. 1 and can comprise robotic arm 120 and the like.

Display device 1006 can be used to display graphical user interface 1018 to allow an operator to receive output from system 1000 and provide input to system 1000. Graphical user interface 1018 can display three-dimensional virtual models of anatomy of a patient that include patient-specific bone density and strength information along with a model of a selected prosthesis and bone grafts, as well as overlays of the actual donor and graft sites, such as from imaging. Graphical user interface 1018 can allow a surgeon or surgical planner to manipulate the models to plan a surgical procedure. Graphical user interface 1018 can provide feedback on the planned surgical procedure, such as by providing bone graft adequacy, risk assessments, suggested courses of action and the like.

Control system 1008 can comprise a controller as described herein, such as, a robotic controller or computing system 140 of FIG. 1. Memory 1022 can comprise a computer readable storage medium having information related to surgical procedure planning for one or more patients. Memory 1022 can comprise databases of bone models, bone density information, bone stiffness and strength information, prosthesis engineering parameters (e.g., size, weight, dimensions, material properties), numerical modeling equations, kinematic data, prosthesis usage data (e.g., force data for particular activities) and others.

In examples, planning system 1002, surgical system 1004, display device 1006 and control system 1008 can be configured to communicate and exchange data and information with each other. As such, data input into control system 1008 via graphical user interface 1018 (e.g., resection planes, cut depth, prosthesis placement information) can be used by planning system 1002 to operate simulation engine 1010 and simulate surgical outcomes and by artificial intelligence engine 1012 to update simulation engine 1010. Simulation engine 1010 can be configured to perform finite element analysis of three-dimensional bone models having density and various outputs (stress, strain, strength, etc.) data loaded therein. Data generated by planning system 1002 can be shared with tracking system 1014 and robot system 1016 to implement a planned surgical procedure.

As mentioned, planning system 1002 can access memory 1022 to access information (e.g., electronic data encoded in a non-transitory computer storage medium) relating to look-up tables for bone density and strength information of bone density and strength markers, anatomic bone density and strength information for healthy and diseased bone that can be aggregated from bone information of sample populations, force and impact information for joints in performing various activities, size and weight information for various prosthetic implants and devices, and biographic information for patients, as well as numerical modeling algorithms and equations, artificial intelligence and machine learning engines as described herein. Thus, control system 1008 can compute bone density and strength of a specific patient, such as by consulting an imaging of the specific patient, calculate resection planes and fixation feature locations within the patient-specific bone model for a selected prosthesis, determine impact threshold for bone at the resection planes or fixation feature locations, calculate force information for particular activities of the specific patient, provide risk assessment feedback (e.g., potential for bone damage and prosthesis loosening) for the selected bone graft, and provide a recommendation for selecting a bone graft scenario or selecting a different bone graft scenario.

FIG. 11 illustrates a block diagram of an example machine 1100 upon which any one or more of the techniques discussed herein may be performed in accordance with some embodiments. For example, machine 1100 can comprise computing system 140 of FIG. 1. Machine 1100 can comprise an example of a controller for planning system 1002 (FIG. 10). As such instructions 1124 can be executed by hardware processor 1102 to generate and bone density information, implant scenarios for various prostheses, and risk assessments of each implant scenario.

Machine 1100 can operate as a standalone device or may be connected (e.g., networked) to other machines. In a networked deployment, machine 1100 may operate in the capacity of a server machine, a client machine, or both in server-client network environments. In an example, machine 1100 can act as a peer machine in peer-to-peer (P2P) (or other distributed) network environment. Machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine 1100 can comprise a computer system and can include hardware processor 1102 (e.g., a central processing unit (CPU), a graphics processing unit (GPU), a hardware processor core, or any combination thereof), main memory 1104 and static memory 1106, some or all of which may communicate with each other via interlink 1108, which can comprise a bus. Machine 1100 may further include display unit 1110, alphanumeric input device 1112 (e.g., a keyboard), and user interface (UI) navigation device 1114 (e.g., a mouse). In an example, display unit 1110, alphanumeric input device 1112 and UI navigation device 1114 may be a touch screen display. Machine 1100 may additionally include a drive unit or storage device 1116, signal generation device 1118 (e.g., a speaker), network interface device 1120, and one or more sensors 1121, such as a global positioning system (GPS) sensor, compass, accelerometer, or other sensors. Machine 1100 may include output controller 1128, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

Storage device 1116 may include machine readable medium 1122 on which is stored one or more sets of data structures or instructions 1124 (e.g., software) embodying or utilized by any one or more of the techniques or functions described herein, such as the surgical planning tools, bone density and finite element analysis tools, and others. Instructions 1124 may also reside, completely or at least partially, within main memory 1104, within static memory 1106, or within hardware processor 1102 during execution thereof by machine 1100. In an example, one or any combination of hardware processor 1102, main memory 1104, static memory 1106, or storage device 1116 may constitute machine readable media.

While machine readable medium 1122 is illustrated as a single medium, the term “machine readable medium” may include a single medium or multiple media (e.g., a centralized or distributed database, and/or associated caches and servers) configured to store the one or more instructions 1124. The term “machine readable medium” may include any medium that is capable of storing, encoding, or carrying instructions for execution by machine 1100 and that cause machine 1100 to perform any one or more of the techniques of the present disclosure, or that is capable of storing, encoding or carrying data structures used by or associated with such instructions. Non-limiting machine-readable medium examples may include solid-state memories, and optical and magnetic media.

Instructions 1124 may further be transmitted or received over communications network 1126 using a transmission medium via network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, network interface device 1120 may include one or more physical jacks (e.g., Ethernet, coaxial, or phone jacks) or one or more antennas to connect to communications network 1126. In an example, network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by machine 1100, and includes digital or analog communications signals or other intangible medium to facilitate communication of such software.

Bone density databases, bone strength databases and prosthetic implant device databases can be stored in main memory 1104 and accessed by hardware processor 1102. Hardware processor 1102 can also receive input (such as at alphanumeric input device 1112) relating to patient-specific bone models including patient-specific bone density information, which can be stored in main memory 1104. Machine 1100 can execute numerical model simulations of prosthesis fixation and can execute AI/ML engines to evaluate fixation of prostheses in bone. Machine 1100 can provide visual indicia of the risks associated with various implant scenarios on display unit 1110 (e.g., human interface devices 145). Machine 1100 can be configured to display density, strength, stiffness, displacements, force, stress and strain information at display unit 1110 to provide real-time feedback to a user when planning the suitability and fit of a bone graft to predetermined acceptable thresholds for bone grafts, force, stress and strain.

The systems, devices and methods discussed in the present application can be useful in performing bone graft procedures by providing pre-operative analysis as to whether or not a bone graft will be needed and, if a bone graft is needed, if a harvest site for the bone graft will be suitable, thereby reducing intra-operative decision making and eliminating the likelihood of harvesting an unsuitable bone graft. The pre-operative planning can reduce excess trauma to the patient incurred by having to harvest multiple bone grafts and the likelihood of a bone graft providing unsuitable support that can lead to patient discomfort or trauma post-operatively.

Bone density and strength information can be used to identify weak or unhealthy bone to remove and the location of healthy bone for harvesting a bone graft.

In various aspects, the present disclosure can help a surgeon and artificial intelligence or machine learning engines identify weak or less dense bone and strong or more dense bone patterns so that the placement of bone grafts into bone matter can be selected to improve fixation conditions and improve the long-term post operative outcome.

The bone graft represents a surgical procedure within another surgical procedure, adding time, costs and risks to the patients. Knowing if the bone graft is necessary and of sufficient quality and quantity before the operation shifts time from intra-operative to pre-operative, thereby saving critical operation time.

This shift brings multiple advantages.

The surgeon can identify the potential need of a graft, based on CT identification of low bone density. The surgeon plans ahead of the operation if the planned resected bone may be sufficient for a graft, and can plan the surgery accordingly in terms of required instrumentation, time, anesthesia, etc. This reduces surgery planning uncertainty, allowing hospitals to better plan procedures throughout the day.

The bone graft host site can be evaluated, and the volume of bone material required to fill the void can be estimated. If the graft may not be of sufficient quantity or quality, the surgeon can opt for another solution such as allograft, or synthetic material in advance of the operation, and plan accordingly.

If bone that will be resected as part of the planned operation is of sufficient quality and quantity, the surgeon can use that bone instead of harvesting the bone at another anatomical location. For example, during a shoulder replacement, the resected humeral head may be used as bone graft material in that same surgery. This humeral bone could be used as graft material for the humerus, or for the glenoid, or both. In another example, the distal femur or proximal tibia bone material may be used for a primary knee replacement, and avoid the need for cones or augments. The present disclosure allows evaluating if that bone will be sufficient for the specific case, thereby eliminating the need for harvesting the bone at another anatomic location, or using systems to replace missing bone.

The systems, devices and methods of the present application can help reduce the risk of infection, which is still one of the main issues in current surgery. The risk of infection increases with operation time, but also when another surgical procedure needs to be performed at another anatomical location (as in an iliac crest bone graft).

A digital, pre-operative tool fits within the current demand for personalized medicine and services.

Depending on the imaging modality, a full body scan may be available (ex. DEXA). In such case, candidate bone graft harvest surgical sites may be evaluated for that patient, and optimal harvesting sites may be proposed.

In summary, the systems, devices and methods of the present application can help cut down operation time, reduce complications and healthcare costs.

### Examples

Example 1 is a computer-implemented method of pre-operatively evaluating bone structure for use of a bone graft in a surgical procedure, the computer-implemented method comprising: generating a three-dimensional bone model of a bone of a patient for output in a video display unit; determining a volume of interest (VOI) on the three-dimensional bone model where a bone defect exists; adding an internal bone property for bone matter of the bone structure to the three-dimensional bone model at the volume of interest; analyzing the internal bone property in the volume of interest relative to a treatment for a bone defect; and outputting indicia on the video display unit that indicates utility of a bone graft for the treatment.

In Example 2, the subject matter of Example 1 optionally includes D generation using a plurality of two-dimensional x-ray images or aggregating a plurality of three-dimensional scans of the bone.

In Example 3, the subject matter of any one or more of Examples 1-2 optionally include wherein generating the three-dimensional bone model of the bone of the patient comprises generating Dual-energy X-ray absorptiometry (DEXA) imagery.

In Example 4, the subject matter of any one or more of Examples 1-3 optionally include determining that the internal bone property in the volume of interest is insufficient to allow the bone defect to be treated; and digitally assessing bone density of a bone graft harvest site for the internal bone property by comparing bone density from the bone graft harvest site obtained from imaging to database information of bone density or to regions near the bone defect.

In Example 5, the subject matter of Example 4 optionally includes wherein: the bone defect comprises a defective joint surface; and the bone matter comprises cancellous bone.

In Example 6, the subject matter of Example 5 optionally includes wherein adding the internal bone property for bone matter of the bone structure to the three-dimensional bone model at the volume of interest comprises adding three-dimensional bone density information to the three-dimensional bone model.

In Example 7, the subject matter of Example 6 optionally includes wherein adding three-dimensional bone density information to the three-dimensional bone model comprises adding a number representing a grey value of each pixel of the three-dimensional bone model.

In Example 8, the subject matter of any one or more of Examples 6-7 optionally include wherein analyzing the internal bone property in the volume of interest relative to a treatment for a bone defect comprises determining if bone density in the volume of interest is sufficient to allow a medical implant to attach to the bone.

In Example 9, the subject matter of any one or more of Examples 6-8 optionally include wherein analyzing the internal bone property in the volume of interest relative to a treatment for a bone defect comprises determining if a volume of bone paste made from the bone graft harvest site is sufficient to fill the volume of interest.

In Example 10, the subject matter of any one or more of Examples 6-9 optionally include generating density information using a bone density marker or a calibration phantom positioned within imaging of the volume of interest.

In Example 11, the subject matter of any one or more of Examples 6-10 optionally include wherein outputting indicia on the video display unit that indicates utility of the bone graft for the treatment comprises outputting indicia on the video display unit that indicates the internal bone property in the volume of interest.

In Example 12, the subject matter of Example 11 optionally includes wherein outputting indicia on the video display unit that indicates utility of the bone graft for the treatment further comprises comparing the internal bone property in the volume of interest to a scale of various bone densities derived from a bone density database.

In Example 13, the subject matter of any one or more of Examples 4-12 optionally include wherein: the bone defect comprises a bone fracture; and the bone matter comprises cortical bone.

In Example 14, the subject matter of Example 13 optionally includes wherein adding the internal bone property for bone matter of the bone structure to the three-dimensional bone model at the volume of interest comprises adding three-dimensional bone strength information to the three-dimensional bone model.

In Example 15, the subject matter of Example 14 optionally includes wherein adding three-dimensional bone strength information to the three-dimensional bone model comprises performing finite element analysis (FEA) of the three-dimensional bone model in the VOI.

In Example 16, the subject matter of any one or more of Examples 14-15 optionally include wherein analyzing the internal bone property in the volume of interest relative to a treatment for a bone defect comprises determining if bone strength of a bone graft to fill the volume of interest is sufficient to support the bone structure.

In Example 17, the subject matter of any one or more of Examples 13-16 optionally include generating force data to apply to the three-dimensional bone model at the volume of interest that is derived from a publicly available database of physical activities or a musculoskeletal model.

In Example 18, the subject matter of any one or more of Examples 13-17 optionally include wherein outputting indicia on the video display unit that indicates utility of the bone graft for the treatment comprises outputting indicia on the video display unit that indicates the internal bone property in the volume of interest.

In Example 19, the subject matter of Example 18 optionally includes wherein outputting indicia on the video display unit that indicates utility of the bone graft for the treatment comprises comparing the internal bone property in the volume of interest to a scale of various bone strengths derived from a bone density database.

In Example 20, the subject matter of any one or more of Examples 4-19 optionally include wherein analyzing the internal bone property in the volume of interest relative to the treatment for a bone defect comprises determining fit of medical implant at the volume of interest; and adjusting a location of the medical implant or the volume of interest based on the determined utility of the bone graft for the treatment.

In Example 21, the subject matter of any one or more of Examples 1-20 optionally include adjusting a bone modification based on results of analyzing the internal bone property in the volume of interest relative to a treatment for a bone defect.

In Example 22, the subject matter of any one or more of Examples 1-21 optionally include running an artificial intelligence engine to identify weakness in the VOI; and updating the artificial intelligence engine with any identified weakness in the three-dimensional bone model.

Example 23 is a surgical planning system comprising: a controller for a computer-implemented surgical planning system; an output device in communication with the controller; an input device in communication with the controller; and memory having instructions stored therein executable by the controller to generate a surgical plan, the instructions comprising: generate a three-dimensional bone model showing bone structure of a bone of a patient for output in the output device; determine a first volume of interest (VOI) on the three-dimensional bone model where a bone defect exists; determine a second volume of interest (VOI) on the three-dimensional bone model where a potential bone graft exits; add an internal bone property for bone matter of the bone structure to the three-dimensional bone model at the first VOI and the second VOI; analyze the internal bone property in the volume of interest relative to a treatment for a bone defect; and output indicia on the output device that indicates utility of a bone graft for the treatment.

In Example 24, the subject matter of Example 23 optionally includes wherein the instructions further comprise: generate the three-dimensional bone model of the bone of the patient by using 2D to 3D generation using a plurality of two-dimensional x-ray images, by aggregating a plurality of three-dimensional scans of the bone, or by using Dual-energy X-ray absorptiometry (DEXA) imagery.

In Example 25, the subject matter of any one or more of Examples 23-24 optionally include wherein the instructions further comprise: digitally assessing bone density of the second VOI for the internal bone property; wherein: the bone defect comprises a defective joint surface; and the bone matter comprises cancellous bone.

In Example 26, the subject matter of Example 25 optionally includes wherein adding the internal bone property for bone matter of the bone structure to the three-dimensional bone model at the second VOI comprises adding three-dimensional bone density information to the three-dimensional bone model.

In Example 27, the subject matter of Example 26 optionally includes wherein: adding three-dimensional bone density information to the three-dimensional bone model comprises adding a number representing a grey value of each pixel of the three-dimensional bone model; and analyzing the internal bone property in the volume of interest relative to a treatment for a bone defect comprises determining if bone density in the second VOI is sufficient to allow a medical implant to attach to the bone.

In Example 28, the subject matter of any one or more of Examples 25-27 optionally include wherein outputting indicia on the output device that indicates utility of the bone graft for the treatment comprises: outputting indicia on the output device that indicates the internal bone property in the second VOI; and comparing the internal bone property in the second VOI to a scale of various bone densities derived from a bone density database.

In Example 29, the subject matter of any one or more of Examples 23-28 optionally include wherein: digitally assessing bone strength of the second VOI for the internal bone property; wherein: the bone defect comprises a bone fracture; and the bone matter comprises cortical bone.

In Example 30, the subject matter of Example 29 optionally includes wherein adding the internal bone property for bone matter of the bone structure to the three-dimensional bone model at the second VOI comprises adding three-dimensional bone strength information to the three-dimensional bone model.

In Example 31, the subject matter of Example 30 optionally includes wherein: adding three-dimensional bone strength information to the three-dimensional bone model comprises performing finite element analysis (FEA) of the three-dimensional bone model in the VOI; and analyzing the internal bone property in the volume of interest relative to a treatment for a bone defect comprises determining if bone strength of a bone graft to fill the volume of interest is sufficient to support the bone structure.

In Example 32, the subject matter of any one or more of Examples 29-31 optionally include wherein outputting indicia on the output device that indicates utility of the bone graft for the treatment comprises: outputting indicia on the output device that indicates the internal bone property in the second VOI; and comparing the internal bone property in the second VOI to a scale of various bone strengths derived from a bone density database.

Each of these non-limiting examples can stand on its own, or can be combined in various permutations or combinations with one or more of the other examples.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A computer-implemented method of pre-operatively evaluating bone structure for use of a bone graft in a surgical procedure, the computer-implemented method comprising:
generating a three-dimensional bone model of a bone of a patient for output in a video display unit;
determining a volume of interest (VOI) on the three-dimensional bone model where a bone defect exists;
adding an internal bone property for bone matter of the bone structure to the three-dimensional bone model at the volume of interest;
analyzing the internal bone property in the volume of interest relative to a treatment for a bone defect; and
outputting indicia on the video display unit that indicates utility of a bone graft for the treatment.

2. The computer-implemented method of claim 1, wherein generating the three-dimensional bone model of the bone of the patient comprises 2D to 3D generation using a plurality of two-dimensional x-ray images or aggregating a plurality of three-dimensional scans of the bone.

3. The computer-implemented method of claim 1, wherein generating the three-dimensional bone model of the bone of the patient comprises generating Dual-energy X-ray absorptiometry (DEXA) imagery.

4. The computer-implemented method of claim 1, further comprising:
determining that the internal bone property in the volume of interest is insufficient to allow the bone defect to be treated; and
digitally assessing bone density of a bone graft harvest site for the internal bone property by comparing bone density from the bone graft harvest site obtained from imaging to database information of bone density or to regions near the bone defect.

5. The computer-implemented method of claim 4, wherein:
the bone defect comprises a defective joint surface; and
the bone matter comprises cancellous bone.

6. The computer-implemented method of claim 5, wherein adding the internal bone property for bone matter of the bone structure to the three-dimensional bone model at the volume of interest comprises adding three-dimensional bone density information to the three-dimensional bone model.

7. The computer-implemented method of claim 6, wherein adding three-dimensional bone density information to the three-dimensional bone model comprises adding a number representing a grey value of each pixel of the three-dimensional bone model.

8. The computer-implemented method of claim 6, wherein analyzing the internal bone property in the volume of interest relative to a treatment for a bone defect comprises determining if bone density in the volume of interest is sufficient to allow a medical implant to attach to the bone.

9. The computer-implemented method of claim 6, wherein analyzing the internal bone property in the volume of interest relative to a treatment for a bone defect comprises determining if a volume of bone paste made from the bone graft harvest site is sufficient to fill the volume of interest.

10. The computer-implemented method of claim 6, further comprising:
generating density information using a bone density marker or a calibration phantom positioned within imaging of the volume of interest.

11. The computer-implemented method of claim 6, wherein outputting indicia on the video display unit that indicates utility of the bone graft for the treatment comprises outputting indicia on the video display unit that indicates the internal bone property in the volume of interest.

12. The computer-implemented method of claim 11, wherein outputting indicia on the video display unit that indicates utility of the bone graft for the treatment further comprises comparing the internal bone property in the volume of interest to a scale of various bone densities derived from a bone density database.

13. The computer-implemented method of claim 4, wherein:
the bone defect comprises a bone fracture; and
the bone matter comprises cortical bone.

14. The computer-implemented method of claim 13, wherein adding the internal bone property for bone matter of the bone structure to the three-dimensional bone model at the volume of interest comprises adding three-dimensional bone strength information to the three-dimensional bone model.

15. The computer-implemented method of claim 14, wherein adding three-dimensional bone strength information to the three-dimensional bone model comprises performing finite element analysis (FEA) of the three-dimensional bone model in the VOI.

16. The computer-implemented method of claim 14, wherein analyzing the internal bone property in the volume of interest relative to a treatment for a bone defect comprises determining if bone strength of a bone graft to fill the volume of interest is sufficient to support the bone structure.

17. The computer-implemented method of claim 13, further comprising:
generating force data to apply to the three-dimensional bone model at the volume of interest that is derived from a publicly available database of physical activities or a musculoskeletal model.

18. The computer-implemented method of claim 13, wherein outputting indicia on the video display unit that indicates utility of the bone graft for the treatment comprises outputting indicia on the video display unit that indicates the internal bone property in the volume of interest.

19. The computer-implemented method of claim 18, wherein outputting indicia on the video display unit that indicates utility of the bone graft for the treatment comprises comparing the internal bone property in the volume of interest to a scale of various bone strengths derived from a bone density database.

20. The computer-implemented method of claim 4, wherein analyzing the internal bone property in the volume of interest relative to the treatment for a bone defect comprises
determining fit of medical implant at the volume of interest; and
adjusting a location of the medical implant or the volume of interest based on the determined utility of the bone graft for the treatment.

21. The computer-implemented method of claim 1, further comprising adjusting a bone modification based on results of analyzing the internal bone property in the volume of interest relative to a treatment for a bone defect.

22. The computer-implemented method of claim 1, further comprising:
running an artificial intelligence engine to identify weakness in the VOI; and
updating the artificial intelligence engine with any identified weakness in the three-dimensional bone model.

23. A surgical planning system comprising:
a controller for a computer-implemented surgical planning system;
an output device in communication with the controller;
an input device in communication with the controller; and
memory having instructions stored therein executable by the controller to generate a surgical plan, the instructions comprising:
generate a three-dimensional bone model showing bone structure of a bone of a patient for output in the output device;
determine a first volume of interest (VOI) on the three-dimensional bone model where a bone defect exists;
determine a second volume of interest (VOI) on the three-dimensional bone model where a potential bone graft exits;
add an internal bone property for bone matter of the bone structure to the three-dimensional bone model at the first VOI and the second VOI;
analyze the internal bone property in the volume of interest relative to a treatment for a bone defect; and
output indicia on the output device that indicates utility of a bone graft for the treatment.

24. The surgical planning system of claim 23, wherein the instructions further comprise: generate the three-dimensional bone model of the bone of the patient by using 2D to 3D generation using a plurality of two-dimensional x-ray images, by aggregating a plurality of three-dimensional scans of the bone, or by using Dual-energy X-ray absorptiometry (DEXA) imagery.

25. The surgical planning system of claim 23, wherein the instructions further comprise:
digitally assessing bone density of the second VOI for the internal bone property;
wherein:
the bone defect comprises a defective joint surface; and
the bone matter comprises cancellous bone.

26. The surgical planning system of claim 23, wherein:
digitally assessing bone strength of the second VOI for the internal bone property.
